(19) United States
(12) Patent Application Publication
(10) Pub. No.: US 2024/0350056 A1
(43) Pub. Date: Oct. 24, 2024

(54) URINARY CONDITION TREATMENT DEVICE AND SYSTEM PLATFORM

(71) Applicant: CLEARTRAC TECHNOLOGIES, LLC, Elizabethton, TN (US)

(72) Inventors: Brent Laing, Greenwood Village, CO (US); John Green, Elizabethton, TN (US); David Gilliam, Elizabethton, TN (US); Britton Garrett, Elizabethton, CO (US); Keith C Moore, Elizabethton, TN (US)

(21) Appl. No.: 18/685,063

(22) PCT Filed: Aug. 17, 2022
(86) PCT No.: PCT/US2022/040595

§ 371 Date: Feb. 20, 2024

Related U.S. Application Data
(60) Provisional application No. 63/233,868, filed on Aug. 17, 2021.

(51) Int. Cl. A61B 5/20; A61B 5/00; G16H 10/60

(52) U.S. Cl. CPC A61B 5/208; A61B 5/0004; A61B 5/4836; G16H 10/60; A61B 2560/0431; A61B 2562/0223

(57) ABSTRACT

A device and system for treating lower urinary tract symptoms is disclosed. In one embodiment, the system includes a uroflowmeter configured to collect first user data related to a urinary characteristic of the user. The first user data is collected prior to the user receiving an intervention for the lower urinary tract symptoms. The system includes a processing element associated with a medical provider device. The processing element is configured to: receive the first user data; generate a user study, including monitoring protocol, based at least in part on the first user data; receive, from the uroflowmeter, second user data related to the urinary characteristic of the user collected in response to the monitoring protocol; compare the second user data relative to the monitoring protocol; determine a difference based on the comparison; generate a communication related to the difference; and transmit the communication to a user device.

### CROSS-REFERENCE TO RELATED APPLICATIONS

The present application claims priority to U.S. Patent Application No. 63/233,868 filed Aug. 17, 2021 and entitled “Urinary Condition Treatment Device and System Platform,” the entirety of which is incorporated herein by reference for all purposes.

### BACKGROUND

Uroflowmeters are used to monitor and diagnose the urinary tract health of patients. Uroflowmeters measure data regarding the flow of urine during a urination event, or void. Healthcare providers can use data to diagnose obstructions in the urinary tract and other conditions before treatment, and to track treatment progress and effectiveness.

Traditionally, uroflowmeters are bulky, expensive, non-portable devices that remain in doctor's offices. These devices are inconvenient and cannot accurately capture a complete record of patient voids, because patients are put in an un-natural setting, cannot remain in a doctor's office in proximity to the uroflowmeter for a prescribed period more than a few hours, and may produce errant results as patients modify their behavior to use the uroflowmeter. Even if taken home, these uroflowmeters are difficult to use for some people and still may not allow the accurate tracking of a voiding activity.

Related to traditional in-office uroflowmeters, patients may be asked to keep a manual paper or written log, or void diary, of fluid intake and void information, such as urgency, frequency, or volume of urine, in a record, or void (or urinary) diary, of void events over a prescribed period of time. Patients may record voiding volume by voiding into a voiding measurement bowl placed over a toilet. Patients are reluctant to carry the voiding measurement bowl and paper diary with them because the bowl is large, indiscrete, and inconvenient. Due to the lack of portability, there are often voids missing from the diary. Additionally, often there is a delay between a patient completing a void and filling out the corresponding diary entry, which may result in erroneous information being recorded, or missing information. Finally, there is potential for delay in submitting a paper void diary back to the healthcare provider for transcription into an electronic form. Handwriting may be illegible, or worse, the entire diary could be lost. These factors make it difficult for a health care provider to evaluate, diagnose, and track treatment effectiveness. These shortcomings result in delays and reduction in the quality of patient care.

### BRIEF SUMMARY

A system for treating lower urinary tract symptoms is disclosed. The system includes a uroflowmeter configured to collect first user data related to a urinary characteristic of a user. The first user data may be collected prior to the user receiving an intervention for the lower urinary tract symptoms. An intervention may include an action or course of treatment, typically prescribed by a healthcare provider, and may be intended to improve a LUTS, or other medical condition. Some examples of interventions include one or more prescribed courses of drugs, surgery, physical therapy, changes to diet, exercise, combinations thereof, or the like. In some cases, a single intervention may be sufficient to treat a patient's LUTS condition. In some cases, a patient may receive two or more interventions, such as surgery and a course of drugs. The two or more interventions may occur one immediately after the other, or they may be spaced apart to allow for whether an earlier intervention may have been sufficiently effective. The system includes a processing element associated with a medical provider device. The processing element may be configured to receive the first user data; generate a user study, which in some examples may be the basis for a monitoring protocol, based at least in part on the first user data. A monitoring protocol may at least partially define one or more interactions for a patient with a LUTS, or other, condition to have with a voiding device. For example, the monitoring protocol may include the frequency, periodicity, number of times, length of time, time of day, or the like, for which a user is to use a voiding device. A monitoring protocol may also include, separately or additionally, the use of a voiding diary to collect data related to the user's fluid intake, incontinence episodes, exercise, or the like. Data generated from a monitoring protocol, either or both from a voiding device and/or a voiding diary, may be used to prescribe, change, conclude, or adapt an intervention. The processing element may receive, from the uroflowmeter, second user data related to the urinary characteristic of the user collected in response to the monitoring protocol. The processing element may at least in part compare the second user data relative to the monitoring protocol; and the processing element may at least in part determine a difference based on the comparison. The processing element may generate a communication related to the difference. The processing element may at least in part transmit the communication to a user device associated with the user.

The system for treating lower urinary tract symptoms as described herein may be in its entirety, or as a part of, a urologic platform.

Optionally in some embodiments, the uroflowmeter includes a flow chamber configured to receive a flow of urine, a buoyant float positioned within the flow chamber and positionable according to a urine level in the flow chamber, a magnet associated with the buoyant float and positionable according to a position of the buoyant float, and a sensor adjacent to the magnet and configured to detect a movement of the magnet, wherein the movement of the magnet is correlated to the first user data.

Optionally in some embodiments, the communication is configured to prompt the user to follow the monitoring protocol.

Optionally in some embodiments, the uroflowmeter includes a handle portion adapted to be gripped by the user to position the uroflowmeter for a collection of urine from the user.

Optionally in some embodiments, the processing element is configured to generate a remote patient monitoring (“RPM”) record including an entry related to an interaction between the user and the medical provider.

Optionally in some embodiments, the entry includes an RPM time entry configured to track a time of the interaction.

Optionally in some embodiments, the processing element is configured to generate a bill payable by a payer based on the RPM time entry.

Optionally in some embodiments, the urinary characteristic is one or more of a peak urine flow, a time to the peak urine flow from an onset of the urine flow, a urine volume, or a urination time.

Optionally in some embodiments the RPM record is displayed in a user interface that includes a study progress indicator configured to indicate a progress of the user study.

A method for treating lower urinary tract symptoms is disclosed. In some embodiments the method includes receiving, by a uroflowmeter, first user data related to a urinary characteristic of a user. The first user data is collected prior to the user receiving an intervention for the lower urinary tract symptoms. The method includes determining, by a processing element, a user study including a monitoring protocol, based at least in part on the first user data; receiving, from the uroflowmeter, second user data related to the urinary characteristic of the user collected in response to the monitoring protocol; comparing, by the processing element, the second user data relative to the monitoring protocol; determining, by the processing element, a difference based on the comparison; generating, by the processing element, a communication related to the difference; and transmitting, by the processing element, the communication to a user device associated with the user.

Optionally, in some embodiments the method includes adjusting the monitoring protocol based on the difference.

Optionally, in some embodiments the method includes determining the user instructions based on the lower urinary tract symptoms.

Optionally, in some embodiments the method includes determining, by the user device, qualitative user data associated with the lower urinary tract symptoms; and receiving, by the processing element, the qualitative user data.

Optionally, in some embodiments the qualitative user data is related to one or more of total volume of urine output, fluid intake, bladder leaks, bedtime, or awake time.

Optionally, in some embodiments the method includes generating a voiding study including the qualitative data, the first user data, and the second user data.

Optionally, in some embodiments the method includes determining an intervention configured to treat the lower urinary tract symptoms based on the voiding study.

Optionally, in some embodiments the intervention is one of a first level intervention, a second level intervention, or a third level intervention; the second level intervention is more invasive to a body of the user than the first level intervention; and the third level intervention is more invasive to the body of the user than the second level intervention.

Optionally, in some embodiments the urinary characteristic is one or more of a peak urine flow, a time to the peak urine flow from an onset of the urine flow, a urine volume, or a urination time.

Optionally, in some embodiments the method includes generating a bill payable by a payer based on the RPM time entry.

Optionally, in some embodiments the uroflowmeter includes a handle portion adapted to be gripped by the user to position the uroflowmeter for the collection of urine from the user; a flow chamber configured to receive a flow of urine from the user; a magnet associated with the flow chamber and configured to move in response to the flow or level of urine in the flow chamber; and a sensor adjacent the magnet and configured to detect a movement of the magnet.

Optionally, in some embodiments the uroflowmeter includes an arm connecting the buoyant float and the magnet.

Optionally, in some embodiments the arm and the magnet are connected to one another about a pivot axis; the magnet rotates about the pivot axis in response to movement of the float; and the sensor further detects a change in an angular position of the magnet.

Optionally, in some embodiments the uroflowmeter includes a funnel that directs the flow of urine into a reservoir space of the flow chamber.

Optionally, in some embodiments the funnel produces a smooth flow of urine into the flow chamber.

Optionally, in some embodiments the flow chamber defines an inlet that receives the flow of urine; and an outlet that evacuates urine from the flow chamber at a predetermined rate.

Optionally, in some embodiments the uroflowmeter includes electronics that determine a fill volume of the flow chamber using the movement of the magnet.

Optionally, in some embodiments the uroflowmeter includes a funnel at least partially received within the inlet and having one or more contoured surfaces.

Optionally, in some embodiments the float includes a structural member adapted to prevent the flow of urine from overrunning a top of the float.

A method for treating lower urinary tract symptoms is disclosed. In one embodiment, the method includes receiving, by a uroflowmeter, first user data related to a urinary characteristic of the user. The first user data is collected prior to the user receiving an intervention for the lower urinary tract symptoms. The method includes determining a cause of the lower urinary tract symptoms; determining, based on the cause, an intervention for the lower urinary tract symptoms; and receiving, from the uroflowmeter, second user data related to the urinary characteristic of the user collected in response to the monitoring protocol. The second user data is collected after the user begins receiving the intervention.

Optionally, in some embodiments the method includes receiving, from the uroflowmeter, third user data related to the urinary characteristic of the user; and determining an efficacy of the intervention.

### BRIEF DESCRIPTION OF THE SEVERAL VIEWS OF THE DRAWINGS

FIG. 1 is a simplified schematic of a treatment management system

FIG. 2A is a perspective view of an example of a voiding device suitable for use with the system of FIG. 1.

FIG. 2B is a partially exploded perspective view of the voiding device of FIG. 2A.

FIG. 2C is a section view of an example of a voiding device, taken along line 2C-2C of FIG. 2A.

FIG. 2D is a section view of an example of a voiding device, taken along line 2D-2D of FIG. 2C.

FIG. 2E depicts the voiding device of FIG. 2A in a first configuration.

FIG. 2F depicts the voiding device of FIG. 2A in a second configuration.

FIG. 2G depicts the voiding device of FIG. 2A in a third configuration.

FIG. 3A is an exploded view of an alternative uroflowmeter in accordance with various examples of the present disclosure.

FIG. 3B is a cross-sectional view of the uroflowmeter of FIG. 3A taken along line 3C-3C in accordance with various examples of the present disclosure.

FIG. 3C is a partially exploded view of the uroflowmeter of FIG. 3A illustrating a method of decoupling a flow chamber and a handle of the uroflowmeter in accordance with various examples of the present disclosure.

FIG. 3D is a partial detailed perspective view of an example of an attachment of a flow chamber and a handle of the uroflowmeter of FIG. 3A.

FIG. 4 illustrates a flow diagram of a method of managing treatment using the system of FIG. 1.

FIG. 5 is a flow diagram of a method of managing treatment using the system of FIG. 1.

FIG. 6 is a flow diagram of a method of managing treatment using the system of FIG. 1.

FIG. 7 illustrates an example of patient instructions and messages according to the method of FIG. 5.

FIG. 8 illustrates an example of patient instructions and messages according to the method of FIG. 5.

FIG. 9 illustrates an example of a patient study selection interface suitable for use with the system of FIG. 1.

FIG. 10A is an example of a void flow rate profile suitable to be collected with the system of FIG. 1.

FIG. 10B is an example of an accumulated void volume profile suitable to be collected with the system of FIG. 1.

FIG. 11 illustrates an example of an electronic voiding diary suitable for use with the system of FIG. 1.

FIG. 12 is an example of a void log of a patient suitable for use with the system of FIG. 1.

FIG. 13 is an example of a voiding study of a patient suitable for use with the system of FIG. 1.

FIG. 14 is an example of a navigation interface of the system of FIG. 1 showing active patient studies.

FIG. 15 is an example of a navigation interface of the system of FIG. 1 showing completed patient studies.

FIG. 16 is an example of a navigation interface of the system of FIG. 1 showing patient studies selected for attention.

FIG. 17 is an example of a navigation interface of the system of FIG. 1 showing an inventory of uroflowmeters.

FIG. 18 is an example of a remote patient monitoring log of the system of FIG. 1.

FIG. 19 is an example of a navigation interface of the system of FIG. 1.

FIG. 20 is an example of a voiding record of a patient suitable for use with the system of FIG. 1

FIG. 21 illustrates an example of the components of the devices of the system of FIG. 1.

### DETAILED DESCRIPTION

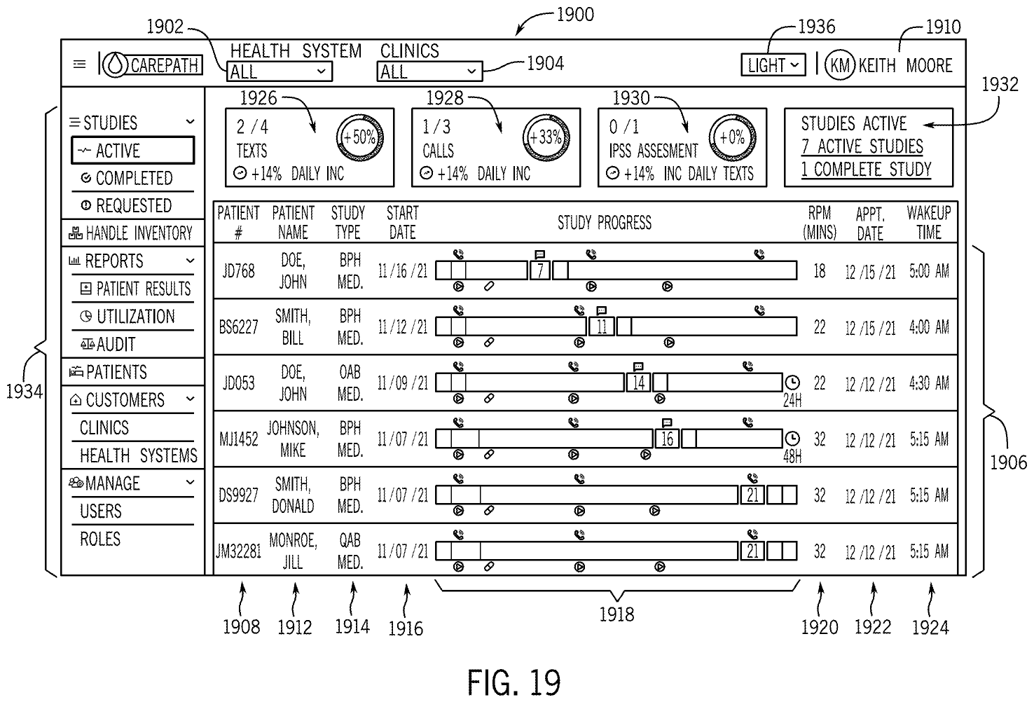

The present disclosure generally relates to systems and methods for analyzing and managing patient data generated from a sensor device to improve accuracy in patient diagnostics and efficacy of treatment. In one example the systems and methods of the present disclosure may be included in a urologic platform. In many implementations, the sensor device is a uroflowmeter or voiding device that measures one or more aspects of a user's urination. In other implementations, patient data may be any data generated about a user's health or biometrics. For example, heart rate, weight, blood pressure, pulse oxidation, sleep, physical activity, and/or other data may be captured by one or more appropriate sensor devices. In one example a voiding device is disclosed that communicates with one more electronic devices (e.g., user smart phone, tablet computer, laptop, server, cloud network, or the like). The system may include a healthcare provider device, a server, a uroflowmeter, and/or a user device. Various devices of the system may be connected to one another via a network such as a private network, a virtual private network, or the internet. The components of the various devices of the treatment management system 100 are discussed with respect to FIG. 19.

In one example, the system is adapted to manage one or more patient studies in the use of a uroflowmeter to diagnose and/or treat lower urinary tract symptoms (“LUTS”) such as benign prostatic hyperplasia (“BHP” or enlarged prostate); overactive bladder (“OAB”); or other urinary diseases or conditions. Based on a patient's use of the uroflowmeter, as compared to a prescribed use, the system may cause a communication, manually or automatically, with a patient to prompt the patient to use a uroflowmeter, provide corrective instructions regarding the use thereof, or generate other patient interactions. The system may also categorize users for further attention from a healthcare provider. The system may provide functionality for a user to record a void diary in conjunction with the use of a uroflowmeter, such as with the use of a user device such as a laptop, tablet, phone, computer, or the like. The system may enable a healthcare provider to manage patient studies for many patients from an interface that categorizes patients based on their usage of the system. The system may record detailed logs of patient interactions. Such logs may simplify the construction of information used in billing of insurance or other payers, and additionally or separately may enable a healthcare provider to monetize functions that have typically been difficult to track and bill for.

A voiding device for use with the system includes positioning and flow sensors that detect void data corresponding to a urinary or use event, such as flow rate, time metadata, and the like. Examples of sensors of the voiding device may include: a buoyant float coupled to a displacement sensor, a temperature sensor, conductivity sensor, opacity sensor, a clock or timer, and the like. The void data detected by the sensors is then verified to determine if it corresponds to a likely void event or whether it corresponds to another non-urinary/void event, such as a patient washing the voiding device after use. This validation helps to prevent irrelevant data from being stored in a patient's void profile information, as well as reduce data transfer within the system, increasing the accuracy of the recorded void profiles to increase treatment effectiveness. As one example, the system may use detected flow characteristics, such as peak flow rate, duration of flow, or total flow volume, to determine if collected data are consistent with a void. The system may validate the data using one or multiple sensors to validate detected data as corresponding to a void event.

In the event that the void data corresponds to an actual urinary user flow event by the patient, the system uses the detected flow information to determine voided urine volume, average urine flow rate, maximum flow rate, voiding duration, voiding flow time, time to maximum urine flow, time of voiding, time between voids, as well as other detected void profile characteristics. The void profile characteristics may be generated in real time or after a series of voids have been collected. The void characteristics or data may be transmitted to a patient's personal electronic device or another electronic device, directly from the voiding device (e.g., Bluetooth®) or via a network, e.g., from a cloud server to the user's device. Similarly, the void characteristics can be transmitted, directly or indirectly, to a healthcare provider and/or third party insurer to assist in treatment, payment and/or research purposes. Because the void profile characteristic may be automatically transferred to a user device and/or a third party or healthcare provider device and the void profile characteristics are captured in real-time as a user is voiding, the data are accurate and can more effectively be used to assess treatment.

Additionally, the disclosure includes methods to correspond detected void characteristics with user activity, fluid consumption, urinary input, or the like to generate a more complete and holistic urinary diary, which was previously not possible with conventional devices. In one example, an application or other program executing on the user device may receive user input related to the user's fluid consumption, urinary events, leakage, and the like, where the user is presented with questions or other interfaces tailored to the detected void profile characteristics. A urinary event is any event involving the flow of urine (including lack of flow) from a user's urinary tract. Using the user input and the void characteristics, the system may generate a void profile for the user for the select use period. Due to the dual-input (user input and detected data), 1:1 correspondence between a void and a user activity can be determined that may increase diagnostic accuracy by a healthcare provider.

Relatedly, the voiding device and user information may be transmitted to a healthcare provider device. For example, the voiding device may be assigned to a particular patient or user from a healthcare provider, and the voiding device may be given a device identifier that corresponds to a patient identifier, associating or linking the two together. From there, the user inputs information into his or her mobile device, and the void data collected by the voiding device then is received, processed, and may be transmitted directly to the healthcare provider device, such that the healthcare provider can receive void profile data, user consumption data, and the like, with the data being tied to the particular patient. After a period of use, e.g., observation or testing period, or when treatment is complete, the user may return the voiding device, or a portion of the voiding device, back to the healthcare provider. At this point, the healthcare provider may dissociate the device from the current user, disconnect (if necessary) a disposable portion of the voiding device (discarding the disposable portion and cleaning/disinfecting the durable portion), recharge the device, and return the durable portion of the voiding device back into service to be assigned or coupled to another user. Disassociating the voiding device may also include recharging the battery, purging stored information, and/or testing the device for readiness.

The voiding device and methods herein may also be used to assess treatment effectiveness, which can then be used to vary treatment, as well as generate payment frameworks for insurers or other third party payers. In one example, the system may receive user pre-treatment void diary data including void characteristics detected by the voiding device as well as user input information and compare the pre-treatment void diary data to data collected during treatment, and/or post-treatment void diary data conducted after the user has completed a prescribed treatment plan. Comparing the results and void characteristics an experienced professional can then help to determine the condition of the users lower urinary tract, prescribe a course of treatment, determine the effectiveness of the treatment, or thresholds (e.g., time to urinary max flow, number of void events, and the like), that may be used to score or otherwise rank the effectiveness of various treatment plans. This scoring can be used to provide feedback to healthcare providers, generate improved treatment plans, and provide payment schedules based on effectiveness. Additionally, if a treatment plan is not effective for particular patients, a healthcare provider or insurer may use the pre-treatment void diary data, data collected during treatment, and/or the post-treatment void diary data to determine the need to prescribe different treatment plans for those patients.

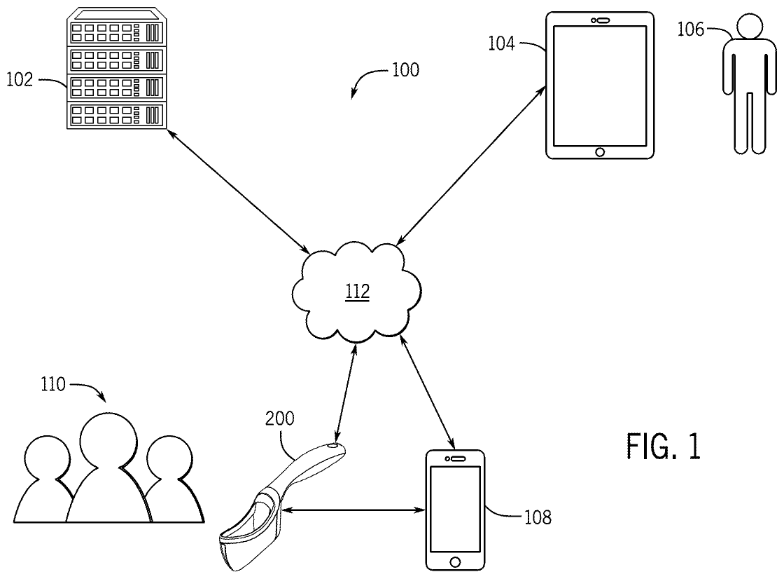

With reference to FIG. 1, in one example the treatment management system 100 includes a server 102, a healthcare provider device 104, a user device 108, and a voiding device 200. In some implementations, one or more devices of the treatment management system 100 may be optional. The devices may be in electrical communication with one another via a network 112.

The healthcare provider device 104 may typically be associated with a healthcare provider 106 such as a system navigator who manages the treatment of patients via the treatment management system 100. The user device 108 may be a device typically associated with a user 110, such as a patient, of the treatment management system 100. Any of the healthcare provider device 104, the user device 108, and/or the server 102 may be an electronic device capable of communicating with one or more other electronic devices. For example, the healthcare provider device 104, the user device 108, and/or the server 102 may be a tablet, phone (e.g., smart phone), laptop, desktop, computer cluster, cloud computing node, or other similar device. The network 112 may be a wired network, a wireless network, or a combination of wired and wireless networks. The network 112 may be a private network, a virtual private network, a public network such as the internet, or combinations thereof.

The healthcare provider 106 may be a healthcare professional such as a nurse, nurse practitioner, nursing assistant, physician, physician's assistant, or the like. The user 110 may be a patient seeking, or under, the care of the healthcare provider 106. Typically, a user 110 using the treatment management system 100 will be seeking care for lower urinary tract symptoms (“LUTS”).

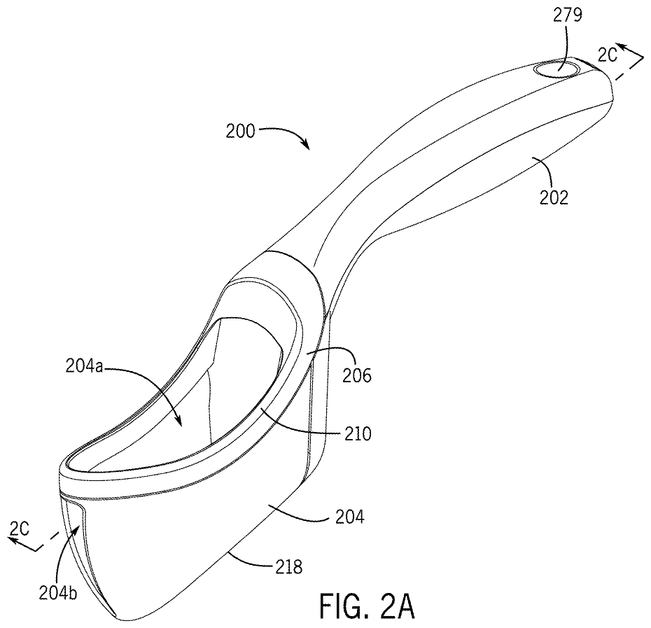

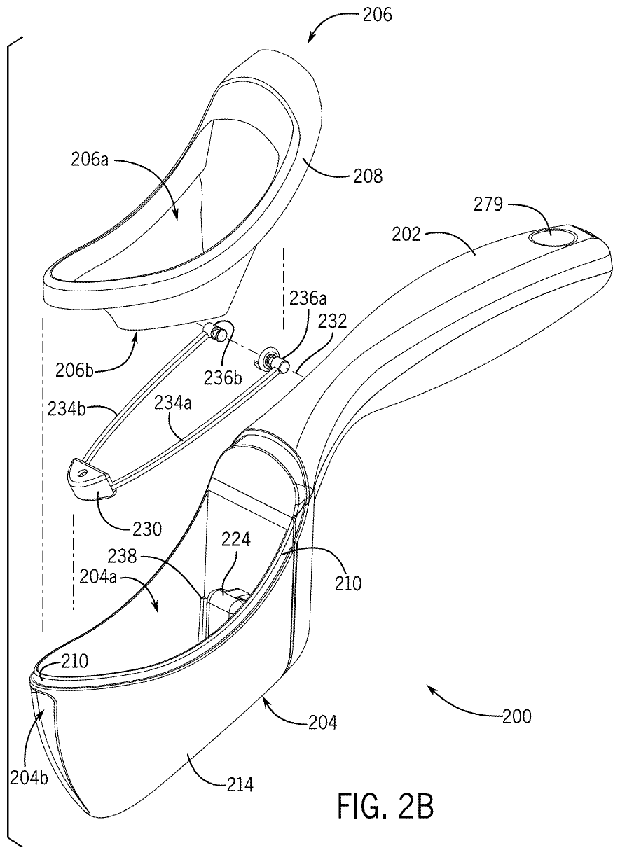

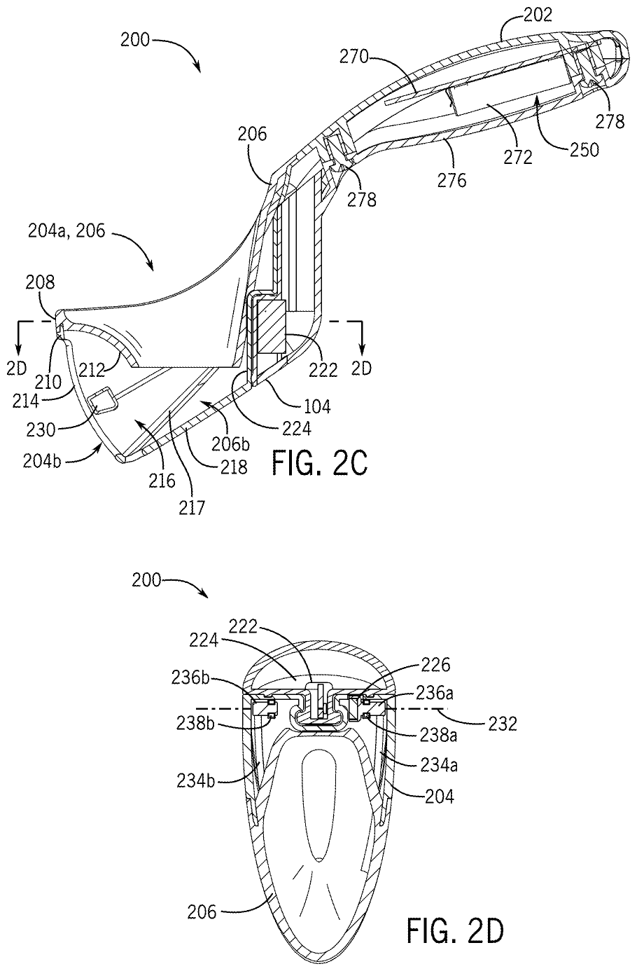

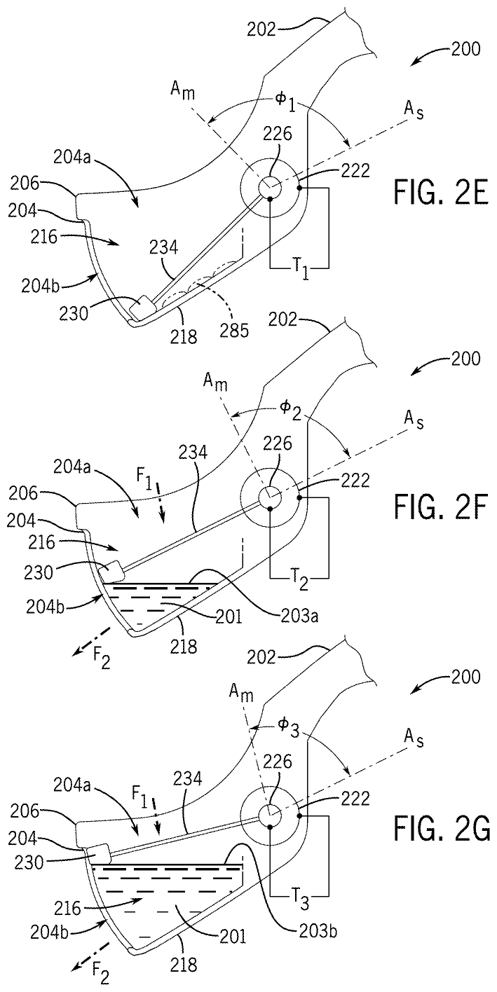

As described in detail with respect to FIG. 2A through FIG. 2G, the voiding device 200 is any device suitable to capture and measure aspects of the urine flow of a user 110. FIG. 2A illustrates a perspective view of an illustrative voiding device 200. FIG. 2B illustrates an exploded view of the voiding device 200 of FIG. 2A. The voiding device 200 includes a flow chamber 204 that momentarily collects and measures urine or other fluid flow while a user is voiding. The flow chamber 204 includes an inlet 204a and an outlet 204b. The flow chamber 204 optionally includes a funnel 206 to produce a smooth flow of urine into the flow chamber. The funnel 206 may define a contour that directs or guides a flow of urine into the flow chamber 204. The contour of the funnel 206 may facilitate reducing turbulent flow of the urine within the flow chamber 204. This may produce a smooth or settled flow of the patient's urine within the flow chamber 204. In some cases, the funnel 206 may form a consistent, laminar flow of the urine. The funnel 206 may include features that orientate or align anatomy of a male patient relative to the voiding device 200 in order to facilitate directing or guiding the male patient's urine into the inlet 204a of the flow chamber 204. The inlet 204a receives urine from the user during use and the outlet 204b allows the collected urine to exit the voiding device 200, such as into a toilet, for disposal. The inlet 204a may be defined along a top of the voiding device 200 to facilitate urine collection. The outlet 204b may be defined along a side (such as a front sidewall as illustrated in FIG. 2A, 2B) of the voiding device 200 to facilitate urine disposal and allow a user to more easily direct the outflow into the proper receptacle. The voiding device 200 typically includes a handle 202 for grasping by a patient. The handle 202 extends from a rearward direction from the flow chamber 204 and may be elongated and relatively slender to provide an ergonomic grip for a patient's hand. In some aspects, a light emitting diode (“LED”) is integrated with the elongated handle. The LED may indicate an orientation value of the voiding device 200 corresponding to a target condition, such as a target orientation value.

The voiding device 200 may include various sensors that detect void data corresponding to a void event. The voiding device 200 may include various types of sensors to determine urine flow rate and urine void volume. In many examples, the voiding device 200 includes one or more flow sensors or fluid level sensors 262. The one or more fluid level sensors 262 may be substantially any type of electronic device, or multiple devices, capable of detecting the fluid level in the flow chamber 204 of the voiding device 200. The fluid level sensor 262 may output an electrical or optical signal corresponding to the level of the fluid in the flow chamber 204. The outputs of the fluid level sensor 262 may correspond to positions of the fluid level sensor 262 during a time interval. The fluid level sensor 262 may include one or more image or optical sensors (e.g., time of flight sensor systems), inductive sensors, magnetic sensors, and/or other sensors. In various examples, the fluid level sensor 262 uses magnetic Hall-effect sensing to determine the fluid level in the flow chamber 204. For example, the fluid level sensor 262 may include a magnetic displacement sensor 222, such as a rotary Hall-effect sensor, that measures the rotary angle of a nearby magnet 226.

It should be noted that the displacement sensor 222 may measure either a linear or an angular displacement of the float. In another example, the fluid level sensor 262 is an accelerometer connected to a flexible float. As the float rises or falls, such as in response to fluid levels, the accelerometer registers a change in its position and thus the fluid level. In another example, the fluid level sensor 262 is a plurality of corresponding pairs wetted electrodes placed at various locations within flow chamber 204. As fluid rises within the flow chamber 204 the fluid may bridge across corresponding pairs of electrodes, enabling a current to flow between them, thereby detecting the fluid level. In another example, the fluid level sensor 262 is a plurality of temperature sensors, such as thermistors, thermocouples, or resistance temperature devices placed at various locations within flow chamber 204. As fluid rises within the flow chamber 204 it may cause a temperature change in various of the plurality of temperature sensors, thereby detecting the fluid level. In another example, the fluid level sensor 262 is a resistive strip that encounters a change in electrical resistance when exposed to an electrically conductive fluid, such as urine. In another example, the fluid level sensor 262 is an optical detector, such as a camera, or light emitter and receiver, that measures liquid level in flow chamber 204 relative to graduation marks (e.g., lines showing the volume of fluid at a given point) within flow chamber 204. In another example, the fluid level sensor 262 is a light emitter and receiver that measure changes in optical transmissive power through a fiber-optic element as that element is exposed to varying levels of fluid within flow chamber 204. In another example, the fluid level sensor 262 is a strain gauge, such as a Wheatstone bridge coupled to a buoyant element. The strain gauge measures the strain on the float as it moves, such as in response to various levels of fluid within flow chamber 204.

The voiding device 200 may have one or more validation sensors 260 that enable the device processing element 252 of the voiding device 200 to analyze one or more validation characteristics to validate whether collected data corresponds to a valid void event. These validation sensors 260 may measure validation characteristics of the voiding device 200 and/or the validation characteristics of the voiding device 200 environment, which can then be compared against typical voiding validation characteristics and/or voiding environments to determine if the event is a void event and if it is a void whether the data is usable (e.g., not too noisy or error prone). In one example, a validation sensor 260 includes one or more of the orientation sensors. In one example, the orientation sensor is an accelerometer. In another example, the orientation sensor is a gyroscope. In one example, the validation sensor 260 detects the grip of a user. In one example, a grip sensor is a capacitive or resistive sensor with an output corresponding to a user's grip. In another example, a validation sensor 260 is a button, switch or the like that the user activates indicating that the voiding device 200 is about to receive a void. In another example, the validation sensor 260 is a proximity sensor, detecting the proximity of the voiding device 200 to the user's hand, body, or genitals, indicating the voiding device 200 as about to receive a void.

The fluid level sensor 262 determines the fluid level in the flow chamber 204. In one example, the fluid level sensor 262 includes a buoyant float 230 coupled with a displacement sensor 222, such that the displacement sensor 222, in combination with the float 230, can be used to determine a level of liquid, or changes to a level of liquid over time within the voiding device 200. The displacement sensor 222 is coupled to the flow chamber 204 and is fluidly sealed from the annular space 216. For example, the flow chamber 204 defines a housing 224 in which the displacement sensor 222 is seated. The housing 224 fluidly seals the displacement sensor 222 from fluid in the flow chamber 204, while permitting the displacement sensor 222 to detect fluid levels in the flow chamber 204.

As illustrated in FIG. 2A-2D, and particularly in FIGS. 2E-2G, for example, the buoyant float positioned within the flow chamber may be positionable according to a urine level in the flow chamber. A magnet associated with the buoyant float may be positionable according to a position of the buoyant float. A sensor may be positioned adjacent to the magnet and configured to detect a movement of the magnet. The movement of the magnet may be correlated to the first user data. For example, the movement of the magnet may be correlated to a flow rate of urine, an amount of urine (e.g., an accumulation of the flow rate over time), changes to the flow rate and or amount, and/or other characteristics of urine flow. As shown for example in FIGS. 2A-2G, as the level of urine or other fluid increases in the flow chamber 204, the float 230 rises within the flow chamber 204. Similarly, as the level of urine decreases in the flow chamber 204, the float 230 falls within the flow chamber 204. The buoyant float may have a density less than that of a liquid such as water, urine, or other bodily fluid such that the buoyant float rests on a surface of the fluid in the flow chamber. In some embodiments, the magnet 226 is located with a pivot axis 232 of an arm 234 that supports the float 230. As the float 230 rises and falls with the fluid level in the flow chamber, the magnet 226 is rotated relative to the displacement sensor 222 via the first and second arms 234a, 234b about a pivot axis 232. The displacement sensor 222 detects an angular position ϕ of the magnet 226 using the magnet's magnetic flux, and the fluid level in the flow chamber 204 can be determined from the angular position data of the magnet 226, e.g., by using a look-up table that correlates the angular position of the magnet 226 to the position of the float 230, and thus the fluid level in the flow chamber 204.

To illustrate the foregoing, FIGS. 2E-2G show the displacement sensor 222 having a reference direction Aₛ and the magnet 226 having a reference direction Aₘ. For purposes of illustration, the angular position ϕ of the magnet 226 may be defined as an angle bounded by the reference direction Aₛ and the reference direction Aₘ. As the fill level in the flow chamber 204 increases, the magnet 226 rotates, and as such, the reference direction Aₘ moves relative to the reference direction Aₛ, thereby indicating a change in the angular position ϕ of the magnet 226.

FIGS. 2E-2G show the displacement sensor 222 detecting a distinct magnetic characteristic T for different angular positions of the magnet 226. For example, in the first configuration of FIG. 2E, the displacement sensor 222 may detect a magnetic characteristic T₁, which may correspond to the magnetic flux exhibited by the magnet 226 when arranged at an angular position ϕ₁. The angular position ϕ₁ may correspond to a position of the float 230 at a bottommost portion of the flow chamber 204, such as when the flow chamber 204 is empty.

As the flow chamber 204 fills with a fluid (e.g., urine), such as generally from the flow path F₁, the float 230 rises, thereby rotating the magnet 226 and allowing the magnet 226 to exhibit a different magnetic characteristic detectable by the displacement sensor 222. To illustrate and with reference to FIG. 2F, the voiding device 200 is shown in a second configuration in which the flow chamber 204 includes urine 201 at a fill level 203a. The float 230 is shown in FIG. 2F in an elevated position from that of FIG. 2E, which corresponds to the fill level 203a of the urine 201. The elevated position of the float 230 at the fill level 203a causes the magnet 226 to rotate for arrangement at an angular position ϕ₂. At the angular position ϕ₂ the magnet 226 may exhibit a magnetic characteristic T₂ detectable by the displacement sensor 222. In this regard, the displacement sensor 222 detects the magnetic characteristic T₂, which may in turn be used by the voiding device 200 (or server, or associated system or device) to determine a fill level of the flow chamber 204 being the fill level 203a shown in FIG. 2F.

As the flow chamber 204 continues to fill with fluid, such as generally from the flow path F₁, the float 230 may continue to rise, thereby further rotating the magnet 226 and allowing the magnet 226 to exhibit a different magnetic characteristic detectable by the displacement sensor 222. To illustrate and with reference to FIG. 2G, the voiding device 200 is shown in a third configuration in which the flow chamber 204 includes urine 201 at a subsequent fill level 203b. The float 230 is shown in FIG. 2G in an elevated position from that of FIG. 2F, which corresponds to the subsequent fill level 203b of the urine 201. The elevated position of the float 230 at the subsequent fill level 203b causes the magnet 226 to rotate for arrangement at an angular position ϕ₃. At the angular position ϕ₃ the magnet 226 may exhibit a magnetic characteristic—T₃ detectable by the displacement sensor 222. In this regard, the displacement sensor 222 may detect the magnetic characteristic T₃, which may in turn be used by the voiding device 200 (or associated system or device) to determine a fill level of the flow chamber 204 being the subsequent fill level 203b shown in FIG. 2G.

The urinary flow rate of the patient can be determined using the fluid level information (e.g., by calculating changes in the fluid level based on a given outflow rate out of the flow chamber 204, such as the outflow rate of flow along the flow path F2). The fluid level also can be converted to a total volume collected by the voiding device 200 (e.g., by integrating the flow rate curve over the total time period of patient use), or in other words the total volume of urine evacuated or voided by the patient. The fluid level, in addition to pitch and/or roll values, detected by validation sensor 260 are used as inputs to a multi-dimensional lookup table to determine retained volume and outflow rate. For example, the calculation/process may be: (pitch, roll (optional), fluid level)=>[lookup table]=>(retained volume, outflow rate).

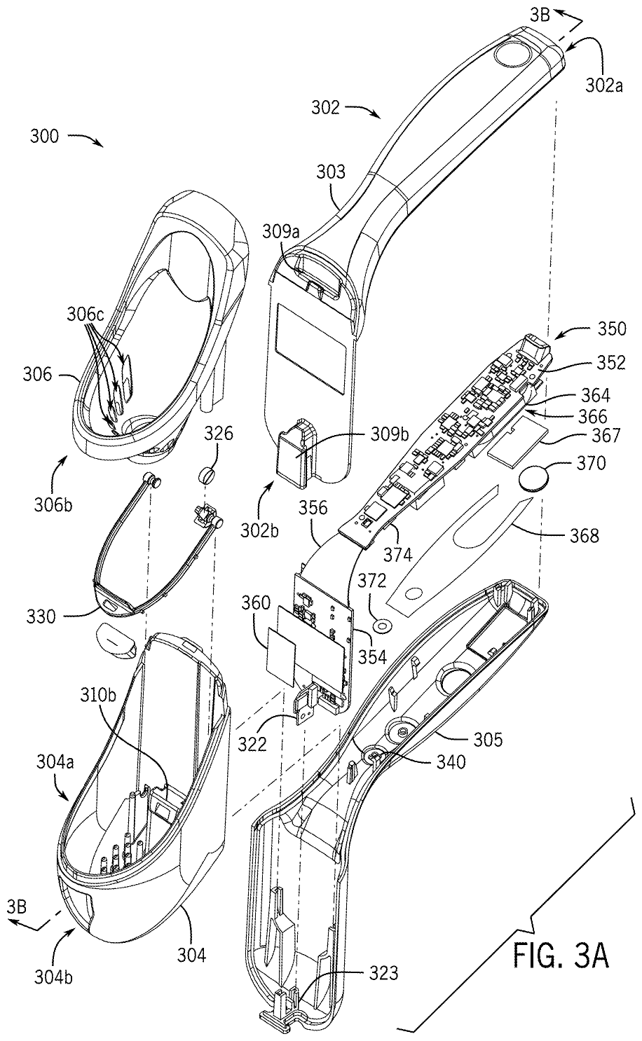

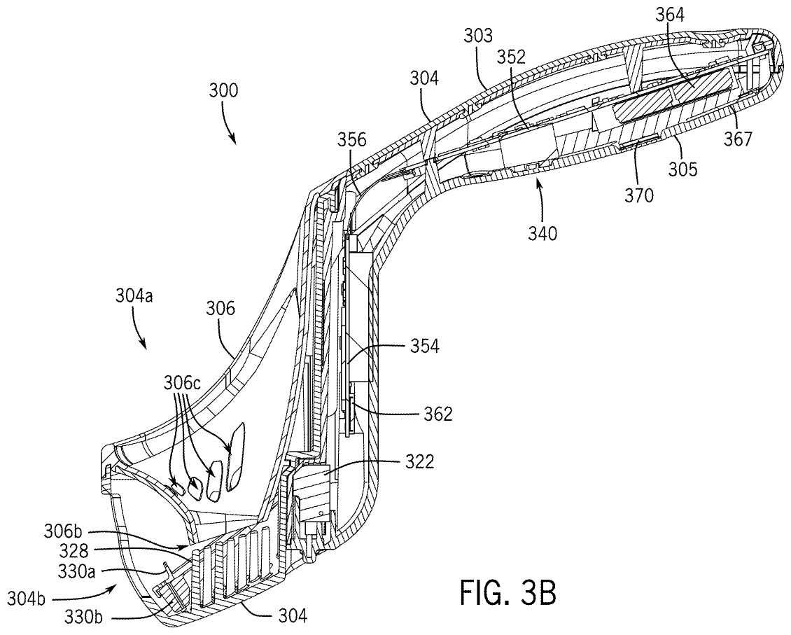

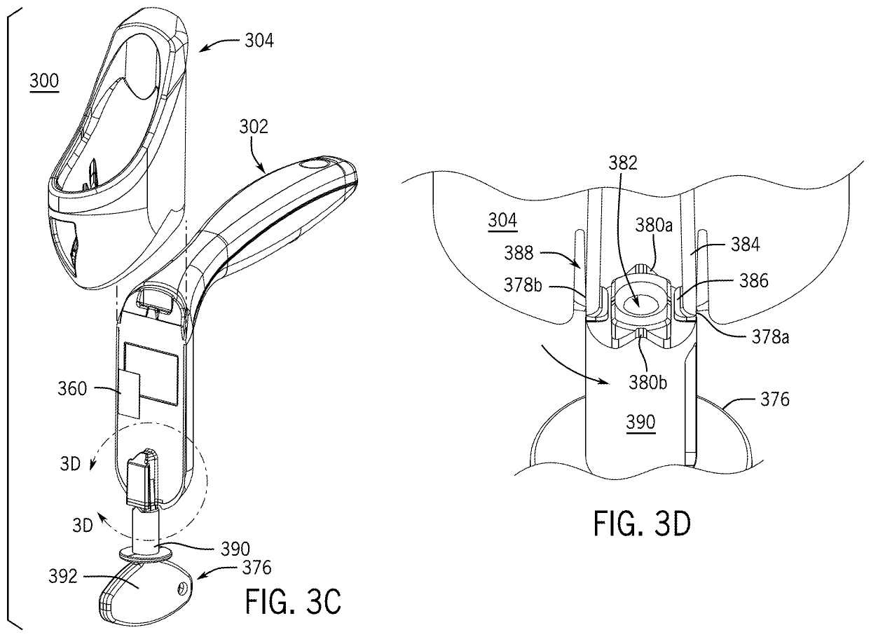

With reference to FIG. 3A-FIG. 3D, a uroflowmeter or voiding device 300 is shown. FIG. 3A is an exploded view of the uroflowmeter 300, and FIG. 3B is a cross-sectional view of the uroflowmeter 300, taken along line 3B-3B of FIG. 3A. FIG. 3C is a partial exploded view of the uroflowmeter 300 of FIG. 3A. FIG. 3D is a partial detailed view of the attachment between the flow chamber 304 and the handle 302 of the uroflowmeter 300.

As illustrated in FIG. 3A-FIG. 3D, the uroflowmeter 300 includes a handle 302, a bowl, bucket, or flow chamber 304, and a funnel 306. The funnel 306 includes one or more funnel outlets to allow fluid to pass from the funnel 306 into the flow chamber 304. The funnel outlets may be of any suitable shape and number to allow a smooth flow of fluid from the funnel 306 into the flow chamber 304. The funnel outlets may be in one configuration for male patients, and in a different configuration for female patients. In one example, the funnel 306 has a primary funnel outlet 306b. In another example, the funnel 306 includes one or more secondary outlets 306c. In one example, the funnel includes five secondary outlets.

The uroflowmeter 300 generally includes the same or similar components and operates in the same or similar manner as the uroflowmeter 200, and thus the descriptions of the uroflowmeter 200 are applicable to the uroflowmeter 300. In this regard, substantially analogous to the examples of the uroflowmeter 200 described above, the uroflowmeter 300 of FIG. 3A-FIG. 3D further includes: a proximal portion 302a, a distal portion 302b, a top shell 303, an inlet 304a, an outlet 304b, a bottom shell 305, a handle groove 309a, a handle tongue 309b, a flow chamber tongue, a flow chamber groove 310b, a sensor 322, a sensor receiving feature 323, energy dissipation features 328, a magnet 326, a float 330, a structural member 330a, a buoyant member 330b, a vent 340, electronics 350, a first printed circuit board 352, a second printed circuit board 354, flex connectors 356, an RFID feature 360, a SIM feature 362, a battery 364, an antenna 366, an NFC feature 367, a proximity sensor 368, a charging coil 370, a vent disc 372, other electrical/mechanical components 374; redundant explanation of which is omitted here for clarity. The structural member 330a may be adapted to maintain accurate readings from the sensor 322 during high periods of fluid flow.

FIG. 3C illustrates the voiding device of FIG. 3A, where the flow chamber 304 of the uroflowmeter 300 is removably attached to the handle 302, thereby allowing the flow chamber 304 to be disposed of after patient use. The flow chambers 204, 304 may be disposable. The handles may be reusable and may be returned to the healthcare provider 106 to be sanitized and assigned to new users 110 and/or placed in inventory. In various examples, the uroflowmeter 300 does not include a disposable funnel. As illustrated in FIG. 3C-FIG. 3D the flow chamber 304 may have one or more grasping features 378a and 378b that grasp cooperating features of the handle 302. In one example, the grasping features 378a, 378b are springs including a cantilevered section 384 separated from the body of the flow chamber 304 by a clearance 388. In the example, the grasping features 378a, 378b include a tang 386. When the grasping features 378a, 378b are in a relaxed position, the tangs 386 grasp corresponding features of the handle, preventing a user from decoupling the flow chamber 304 and the handle 302. The flow chamber 304 and the handle 302 may be decoupled with the use of a key 376. The key may be available to medical professionals, and not available to users. The key 376 may include a handle 392 connected to a shaft 390, a pivot recess 382 defined at one of the shaft 390, and one or more decouplers 380a, 380b disposed radially about the pivot recess 382. The one or more decouplers 380a, 380b cooperate with the one or more grasping features 378a, 378b to allow a medical professional to decouple the flow chamber 304 and the handle 302 of the uroflowmeter 300. In one example, a medical professional inserts the key 376 into the uroflowmeter 300 such that the pivot recess 382 cooperates with a pin in the uroflowmeter 300. The medical professional may rotate, or twist the key 376, causing the one or more decouplers 380a, 380b to press against the one or more grasping features 378a, 378b, flexing the cantilevered section 384 and causing the tang 386 to disengage from the handle 302. The medical professional may then slide the flow chamber 304 away from the handle 302. The medical professional may then dispose of, or disinfect and process for reuse, the flow chamber 304. The medical professional may then reprocess the handle 302 for reuse as previously described. The key 376 and associated features of the handle 302 that prevent user decoupling of the handle 302 and the flow chamber 304 are shown with respect to the example of the voiding device 300, in FIG. 3C-FIG. 3D for example and illustration purposes. The key and these or similar features are equally applicable to, and may be included in, any voiding device disclosed herein, including the voiding device 200 or 300. In one example, the voiding device 200 may be adapted for use by a female patient and the voiding device 300 may be adapted for use by a male patient.

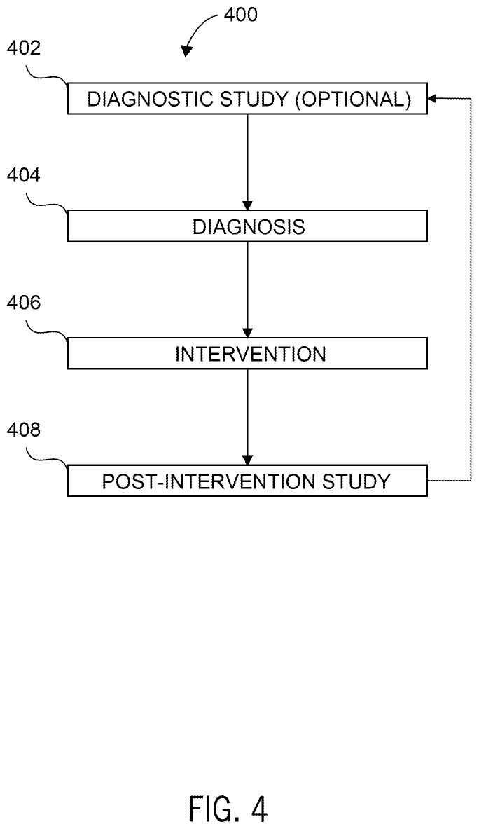

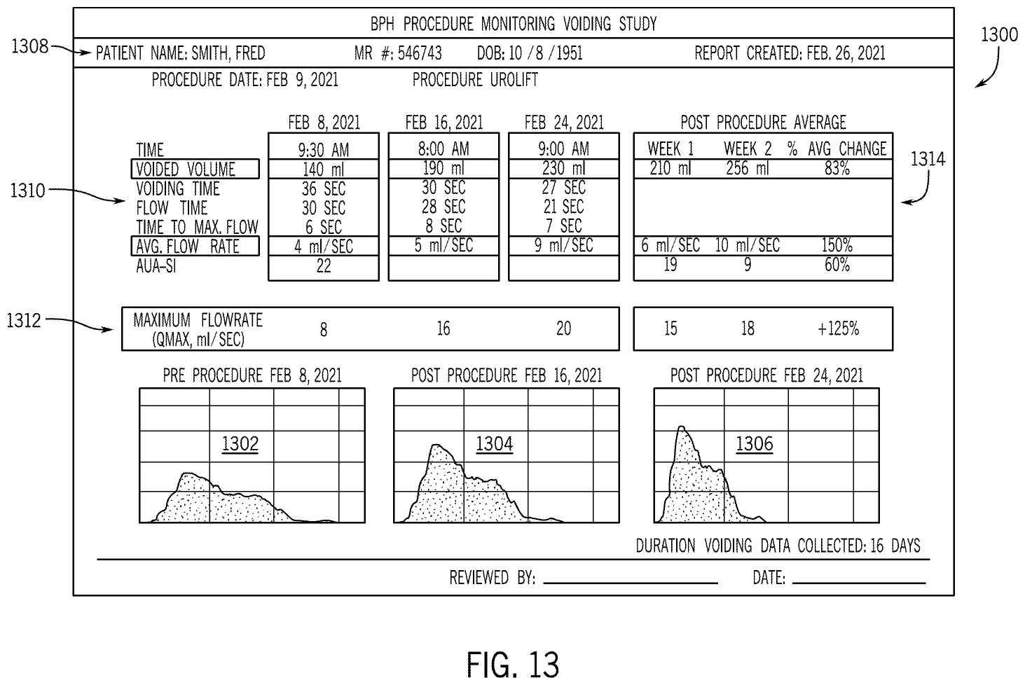

FIG. 4 illustrates a flow diagram of a method of managing treatment using, for example, the system of FIG. 1. The method 400 may begin in operation 402 and a diagnostic study of the user 110 is performed using a sensor device such as the voiding device 200 or the voiding device 300. For example, the user 110 may be prescribed to use the voiding device 200 for a prescribed time period (e.g., two days). In some implementations, the operation 402 may be optional. The voiding device 200 may collect patient data to establish a baseline of urinary health properties of the patient prior to an intervention. The patient data collected in the operation 402 may be used by the treatment management system 100 to generate all or a portion of a user 110 monitoring study. See, e.g., the pre-procedure baseline data 1302 of the voiding study 1300 shown for example in FIG. 13. The operations of the method 400 may be executed in series, in parallel, or in an order other than as shown, and some operations may be optional.

The method 400 may proceed to the operation 404 and the healthcare provider 106 determines a diagnosis of the condition, or likely condition, behind the user's LUTS. For example, many LUTS conditions may have similar clinical presentations. By gathering baseline patient data in the operation 402, and interpreting a voiding study, the healthcare provider 106 may be able to more easily and accurately diagnose the user's condition, which may direct the prescribed treatment.

The method 400 may proceed to the operation 406 and the healthcare provider 106 prescribes an intervention for the user 110, in one example as based upon the baseline data collected in 402 and the resulting diagnosis of 404. An intervention may be a discrete action, such as for example a surgical procedure, which may occur over a relatively short time period, such as for example a day. Additionally or alternatively, an intervention may be a series of actions such as for example a process, course of treatment with a medication, physical therapy, diet, bladder training or the like, which may occur over relatively longer period of time. The intervention may be one or more distinct interventions, such as for example, in a progressively invasive range of interventions. For example, the intervention may be a first level intervention that includes changes to the user's behavior such as diet, fluid intake, scheduled urination, bladder training, physical therapy (e.g., pelvic floor muscle exercises), medication, surgery, or the like. Or the intervention may be a subsequent intervention which may be a repeat of the first level intervention or a different intervention (including a second level intervention, such as for example a more invasive action, such as for example surgery). A voiding device may be used at any time before, during, or after one or more interventions. For example a voiding device may be used during an intervention process that spans over a length of days, weeks, or months to determine the efficacy of the intervention process. A voiding device may be used to change an intervention process that is not achieving desired results, proceed to a more invasive intervention, or to assess that an intervention was successful.

In one implementation, the treatment management system 100 may use the voiding device 200 or the voiding device 300 as a therapeutic device, such as a bladder training device specifically for training the bladder to extend the time between voids. In such cases, the monitoring protocol and the intervention may be at least partially combined, such that the voiding device 200, 300 is used to both monitor and treat the patient. For example, the treatment management system 100 may prompt the user 110 to void the user's bladder on a schedule, optionally using the voiding device 200 or 300. The treatment management system 100 may gradually lengthen the time between notifications to train the user's bladder to hold urine for a longer time. The voiding device 200, 300 may provide feedback regarding the training schedule by gathering data related to the user's voiding characteristics noted above, and indicate any progress toward improvement in extending the period between voiding.

The method 400 may proceed to the operation 408 and the healthcare provider 106 prescribes a post-intervention study of the patient with a sensor device, for example such as the voiding device 200. The post-intervention study may involve the collection of patient data such as voiding data using the voiding device 200. The patient data collected in the operation 402 and the post-intervention data collected in the post-intervention study may be included in a void study. See, e.g., the post-intervention data 1304 and post-intervention data 1306 in the voiding study 1300 shown for example in FIG. 13. Such a voiding study may clearly show whether the LUTS condition of the user 110 has improved, worsened, or stabilized.

The method 400 may return to any of the operation 402, operation 404, and/or operation 406. In particular, if an intervention in operation 406 is not deemed effective based on the patient data collected in the operation 408, progressively more invasive interventions may be prescribed in subsequent executions of the operation 406. For example, a second level intervention such as medication, and/or a third level intervention such as surgery may be prescribed by the healthcare provider 106.

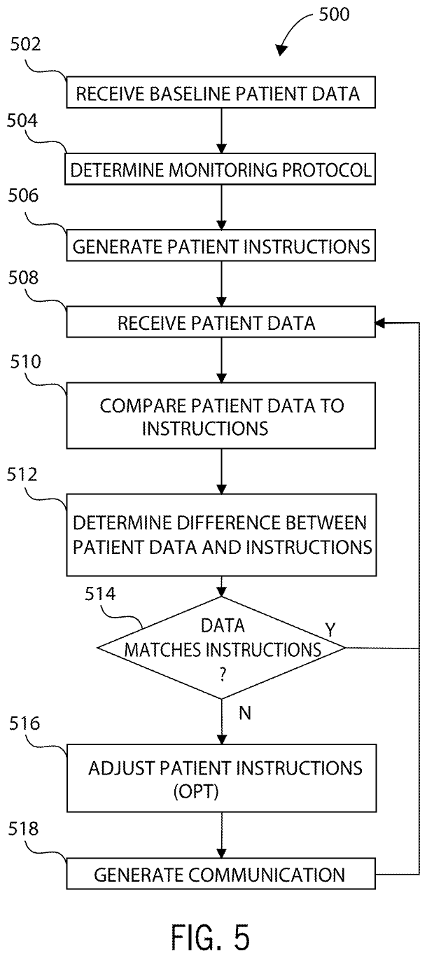

FIG. 5 illustrates a flow diagram of a method of managing treatment using the system of FIG. 1. The operations of the method 500 may be executed in series, in parallel, or in an order other than as shown; and some operations may be optional. The method 500 may begin in operation 502 and the treatment management system 100 receives baseline urinary data from the user 110. For example, the user 110 may be assigned a voiding device 200 to use to record void information as a part of the user's 110 normal day. The baseline data may be received by the treatment management system 100 such as from a user device 108 over the network 112.

For example, the user 110 may take the voiding device 200 to home, work, or other places that the user normally visits in a typical day. An advantage of allowing a user 110 to use a voiding device 200 in their natural setting, rather than in an artificial clinical setting (e.g., doctor's office) is that the user's voiding behavior will be more regular, and presumably more accurately representative of the user's LUTS condition than when under the influence of a clinical setting. For example, users 110 who record voids in a clinical setting may be instructed to abstain from voiding prior to the office visit and may then upon arrival at the office have an urgent need to urinate, due to an excess of stored urine in the bladder, thus influencing results.

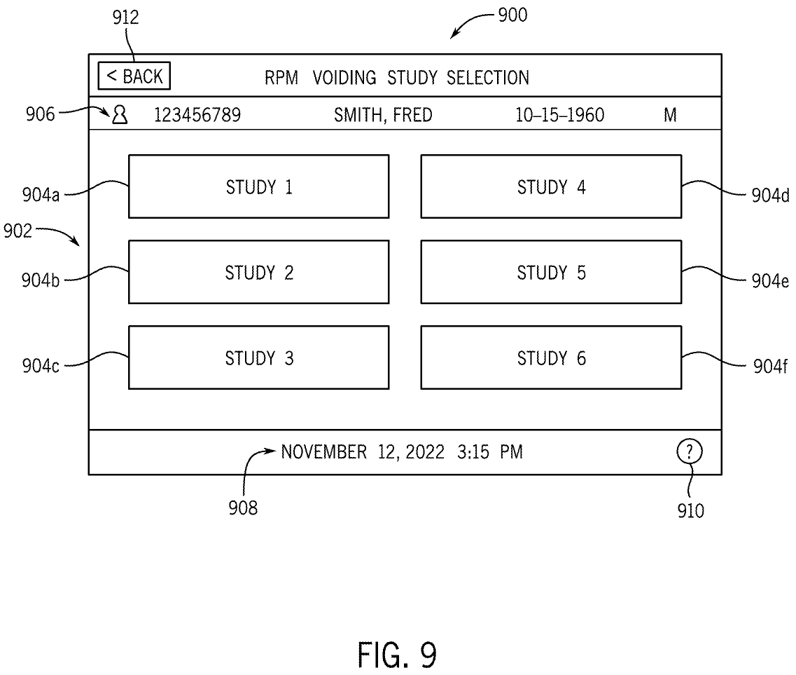

The method 500 may proceed to operation 504 and the healthcare provider 106 may interpret the baseline urinary data and determine a type of monitoring protocol or patient study to prescribe for the user 110. As used herein a patient study may be a period of time in which the user 110 is prescribed to interact with the treatment management system 100 in a manner defined by instructions related to a monitoring protocol from the system, or by the healthcare provider. As discussed further with respect to FIG. 9, patient studies may be adapted to diagnose and/or treat specific conditions, such as BPH and/or OAB. See, e.g., FIG. 9 where the treatment management system 100 presents a list of potential studies for the healthcare provider 106 to prescribe for the user 110 to follow.

The method 500 may proceed to operation 506 and the treatment management system 100 generates patient instructions based on the prescribed study or monitoring protocol. The treatment management system 100 may automatically transmit patient instructions to the user 110 such as via the user device 108 or other means. For example, the treatment management system 100 may instruct the user 110 to use the voiding device 200 to record void data for a prescribed time period (e.g., 8 hours, 24 hours, 2 days, a week, a month, or the like). Additionally, or alternately, the instructions may request that the user records a number of voids with the voiding device 200 for example, the user may be requested to record one, two, three, four, five or more voids with the voiding device 200. Such instructions may be combined. For example, the user may be instructed to record a certain number of voids in a specified time period (e.g., three voids in eight hours). The user may also be instructed to provide additional information about their voiding by noting comment for use by the treatment management system. For instance, the user may make notes on the user device 108 in a separate application, which notes are then communicated with the treatment management system for possible inclusion with the data collected by the voiding device 200.

The method 500 may proceed to operation 508, where the treatment management system 100 receives void data. For example the voiding device 200 may collect void data and may transmit the void data, for example to the user device 108, to the healthcare provider device 104, and/or to the server 102. A device of the treatment management system 100 that receives the void data from the voiding device 200 may transmit the void data to another device. For example, the voiding device 200 may transmit data directly to the user device 108 via a local network such as Bluetooth and/or Wi-Fi. The user device 108 may then transmit the void data to the server 102 and/or the healthcare provider device 104 via a connection with the network 112.

The method 500 may proceed to operation 510 and the treatment management system 100 compares the patient void data to the instructions. The method 500 may proceed to operation 512, where the system 100 determines, based on the comparison in operation 510 whether the patient data is representative of the defined instructions, or if there are deviations from the defined instructions. For example, if the patient instructions are for the user 110 to use the voiding device 200 to record void data for five voids in a one day period and the user 110 only records one void in a one day period, the treatment management system 100 may note the variation from the defined instructions, and may determine that the defined instructions are not being followed. The comparison may have more sensitivity (e.g., a higher sample rate) where the deviation is more severe. For example, if the user misses one void entry the comparison may have a first sensitivity level, and if the user misses an entire day, the comparison may have a second sensitivity level higher than the first sensitivity level.

The method 500 may proceed to operation 514 where the treatment management system 100 takes action based on the difference or deviation determined in operation 512. If the patient data matches the defined instructions, the method 500 may return to the operation 508 and continue to receive patient data. If the patient data does not match the instructions, the method 500 may proceed to the operation 516 and/or the operation 518.

In operation 516, the treatment management system 100 adjusts the patient instructions for the LUTS using the voiding device 200. Returning to the example above, if the user has only recorded one of five prescribed voids in a one day period, the treatment management system 100 may adjust the user instructions e.g., reduce or increase the number of voids prescribed, or may lengthen the time in which to record the voids (e.g., one day to two days). For example, the user instructions may include actions requested or required of the user, which actions have one or more parameters such as a periodicity, frequency, or number of actions. The system 100 may adjust the parameters of the actions based on the user's voiding behavior. For example, the system 100 may adjust (e.g., increase or decrease) the frequency of prompts sent to the user to use the voiding device. Similarly, the system 100 may adjust the period between actions and/or a sum total of actions. The system 100 may adjust parameters of the actions based on the data representing the behavior from the user, for example whether the user has been compliant or non-compliant, and to the extent non-compliant, the level of non-compliance, of the monitoring protocol, and more specifically the user instructions. For example, if the user is using the voiding device as prescribed, the system 100 may decrease a frequency, and or content, of messages. In another example, if the user is not using the voiding device as prescribed in the monitoring protocol, the system 100 may increase the frequency, and/or content of the messages). In the operation 516, the healthcare provider 106 may optionally review patient data and determine or adjust an intervention for the LUTS based on the patient data, as described with respect to the operation 406 of the method 400.

The treatment management system 100 may provide and/or adjust user instructions to gather post-intervention data that the healthcare provider 106 may use to determine treatment efficacy. For example, the treatment management system 100 may be used to collect baseline patient data before treatment (e.g., in the operation 502) and patient data post-intervention. A healthcare provider 106 may use one or both of those two sets of pre-treatment, during-treatment, and/or post-intervention data to determine if the intervention is working and how well, and if other intervention is recommended. See, e.g., the voiding study 1300 in FIG. 13. In some implementations, the operation 516 may be optional.

The method 500 may proceed to operation 518, where the treatment management system 100 optionally generates a communication that is transmitted to the user 110. The system 100 may generate the communication based on the level of the user's compliance with the user instructions, for example to encourage the user to follow the instructions or to correct incorrect usage of the voiding device. The system 100 may also generate communications including educational information, such as how to use the voiding device, lifestyle changes that may help with LUTS symptoms or the like. For example, the treatment management system 100 may generate a message to prompt the user 110 to collect void data. For example, if the voiding device 200 has not been used for a 24 hour period, the treatment management system 100 may generate a message such as “Collecting a complete and accurate record of your voiding pattern is important to enable your physician to manage your Lower Urinary Tract Symptoms (LUTS). The system noticed that you have not recorded any voids for 24 hours. Please use the voiding device today to get back on the care pathway to relief from your bladder symptoms.” The treatment management system 100 may automatically generate and send such messages to the user 110 by any method suitable to reach the user 110. For example the treatment management system 100 may send a text message, email, video, audio, and/or an automatic phone call to the user device 108. The method 500 may return to operation 508 and the treatment management system 100 continues to receive patient data. The communication to the user may be configured to notify the system 100 that the user received, read, and/or accessed the communication. For example, the communication may be configured to determine whether a user watched a video and/or downloaded content linked in a communication. The system 100 may be configured to receive such notifications automatically, or as a result of a user action. For example, a user may reply to a text or email message indicating that the message was received.

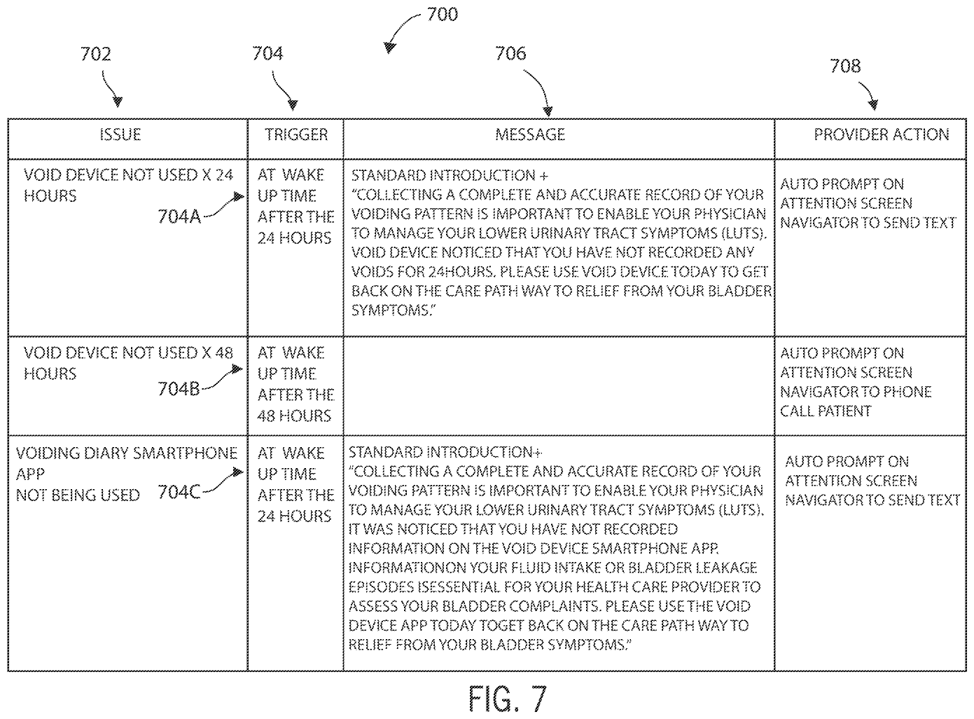

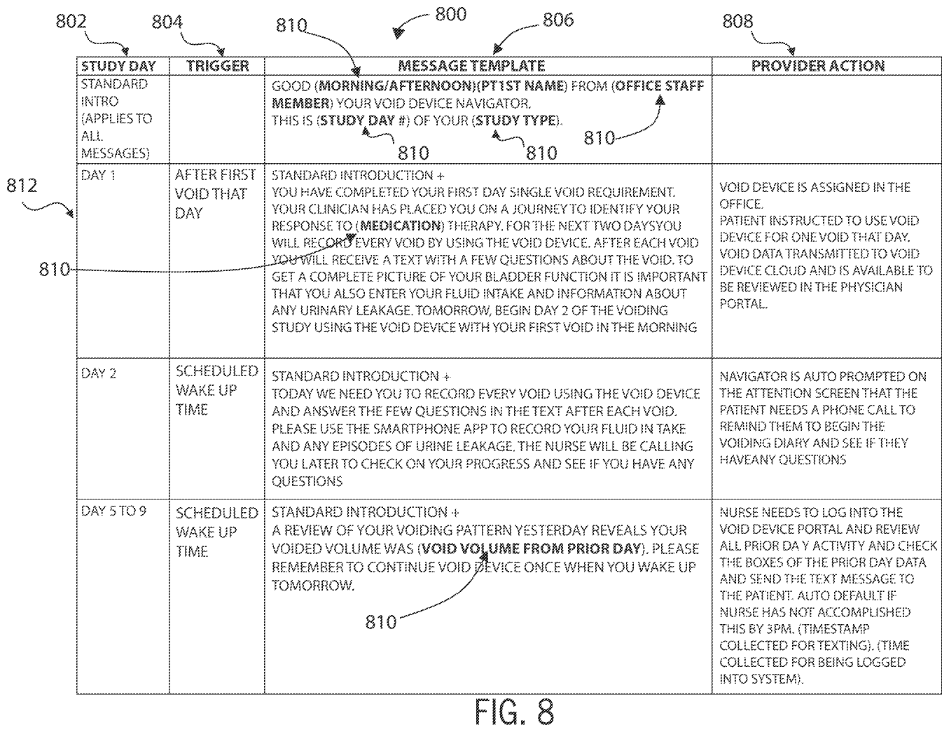

If the patient continues to not comply with, or fails to follow, instructions, the treatment management system 100 may generate additional corrective messages or flags for interaction by the healthcare provider 106 with the user 110. For example, if the user's use of the voiding device does not conform to the user instructions after the system 100 sends a first message, and/or the user does not indicate receipt of the first message from the treatment management system 100, the treatment management system 100 may generate a further message considering the additional information, such as for example a customized message for the user 110, such as “A review of your voiding pattern yesterday reveals your voided volume was (placeholder for user's actual data, such as for example, the void volume from prior day). Please remember to continue to use the void device once when you wake up tomorrow.” As discussed in greater detail with respect to FIGS. 7 and 8, the treatment management system 100 may populate one or more placeholder fields in a standard message to customize the message for a particular user 110 and/or the user's void recording behavior. For example, the system 100 may include a user's name and/or a name of a provider overseeing the user's case. Similarly, the system 100 may customize the message to reflect a number of days the user has been using the voiding device and/or any changes to the user instructions.

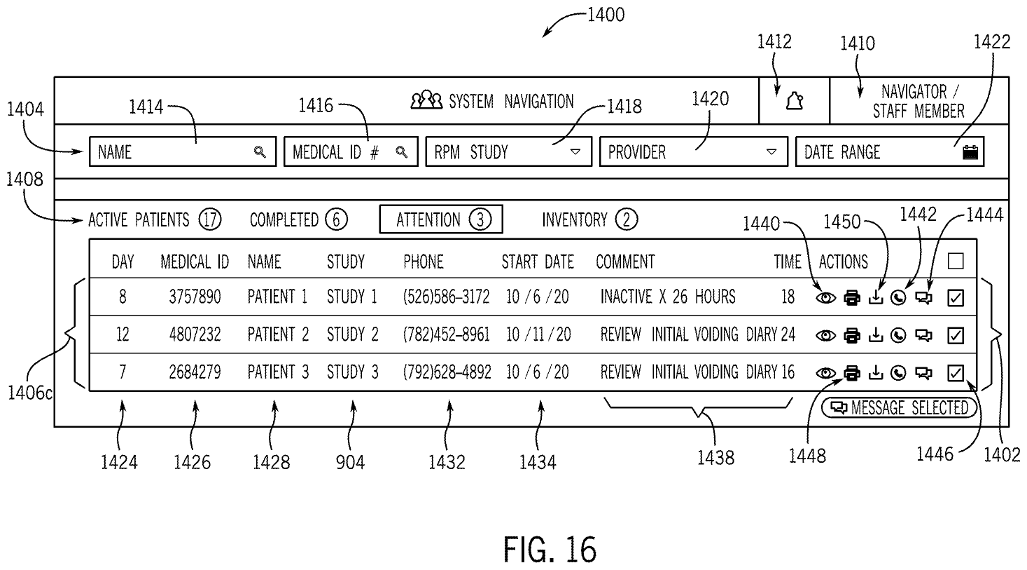

In some implementations, the treatment management system 100 may identify, such as by visually flagging, a user for communication or other follow-up from the healthcare provider 106. For example, if a user consistently fails to use the voiding device 200, the treatment management system 100 may flag or categorize the user in a list of users to receive more frequent or more urgent contact from the system 100, such as by being contacted personally (e.g., called, text messaged, emailed, scheduled for an office visit) by the healthcare provider 106 (see, e.g., FIG. 16). The system 100 may aggregate records of such flagged users in one or more databases and may present such records in a user interface, such as shown in FIG. 16, to allow for convenient and clear attention from the provider 106. Thus, the treatment management system 100 may automatically adjust messaging and/or patient instructions to customize the patient study for each patient, while the study is in progress. The system 100 may customize messages with urgent attention indicators, more persuasive language, bold type and/or capital letters to attempt to persuade the user to follow the user instructions. For example, a customized message to a user who is not following the user instructions, “TODAY WE NEED YOU TO RECORD EVERY VOID USING THE VOID DEVICE AND ANSWER THE FEW QUESTIONS IN THE TEXT AFTER EACH VOID.”

In some implementations, the treatment management system 100 may generate a message that asks the user 110 to use the voiding device 200 and to answer questions about the user's fluid intake and or episodes of urine leakage. For example, the treatment management system 100 may generate a message such as “Today we need you to record every void using the voiding device and answer the few questions after each void. Please use the user device app to record your fluid intake and any episodes of urine leakage. The nurse will be calling you later to check on your progress and see if you have any questions.” See, e.g., FIG. 8.

In some implementations, the method 500 may generate patient communications in operation 518 even when the user 110 is using the voiding device 200 as prescribed by the defined instructions. For example, the treatment management system 100 may generate a message indicating the user's 110 accurate compliance with the defined instructions. For instance, the message may indicate to the user 110 that they are doing a good job and to continue their adherence to the defined instructions and the use of the voiding device 200, which may aid in the continued collection of accurate data for the treatment management system

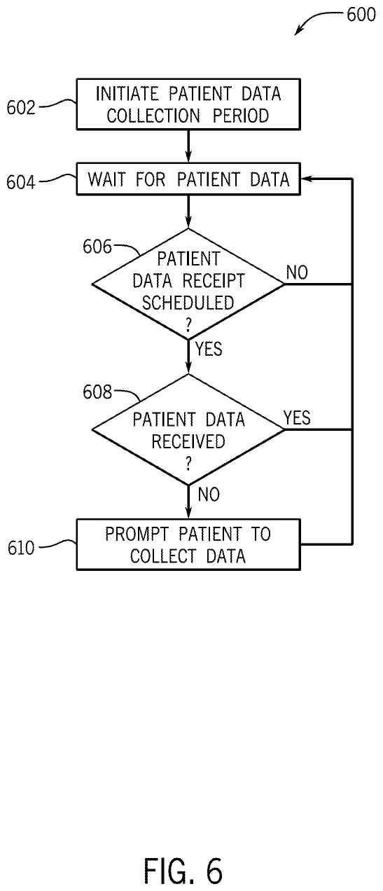

FIG. 6 illustrates a flow diagram of a method of managing treatment using the system of FIG. 1. The method 600 may be a simpler method of managing treatment than the method 500, but may be closely aligned with the method 500 using defined user instructions. For example, the method 600 may be configured to receive patient data when patient data is scheduled for receipt. If the patient data is not received when it is scheduled to be received, the system 100 may prompt the user to collect user data. The operations of the method 600 may be executed in series, in parallel, or in an order other than as shown and some operations may be optional. The method 600 may begin in operation 602, where the treatment management system 100 initiates a patient data collection period. For example, the treatment management system 100 may indicate in a patient record in a database (such as may be stored on the server 102) that the patient is actively collecting urinary data using a voiding device 200.

The method 600 may proceed to the operation 604, where the treatment management system 100 is configured to receive patient urinary data for a certain time. The method 600 may proceed to operation 606 where the treatment management system 100 determines whether receipt of patient data collected from a voiding device 200 is scheduled for a time period. If the patient data is not scheduled to be received, the method 600 may return to the operation 604 and continue to wait for patient data. If patient data is scheduled to be received, the method 600 may proceed to operation 608. In operation 608, the treatment management system 100 determines if patient data is received. If patient data is received, the method 600 may return to the operation 604 and continue to wait for additional patient data. If patient data is not received, the method 600 may proceed to 610 and the treatment management system 100 may prompt the user 110 to collect urinary patient data with the voiding device 200. Operation 610 may be similar to operation 518 described above and may automatically adjust messaging to the patient with either automatic communications, or by prompting a healthcare provider 106 to contact the user.

With reference to FIG. 7 and FIG. 8, specific examples of an implementation of the method 500 are disclosed. FIG. 7 illustrates a patient compliance communication structure 700. Communications may be pre-emptive (e.g., configured to prompt user compliance with the user instructions when the user first begins using the voiding device) and/or may be responsive or corrective (configured to correct non-compliant user usage of the voiding device.) The patient compliance response structure 700 relates issues of patient non-compliance with the defined patient instructions (e.g., as determined in the operation 506 of the method 500), triggers related to the non-compliance, messages adapted to encourage compliance, and recommended healthcare provider 106 actions to help correct the non-compliance.

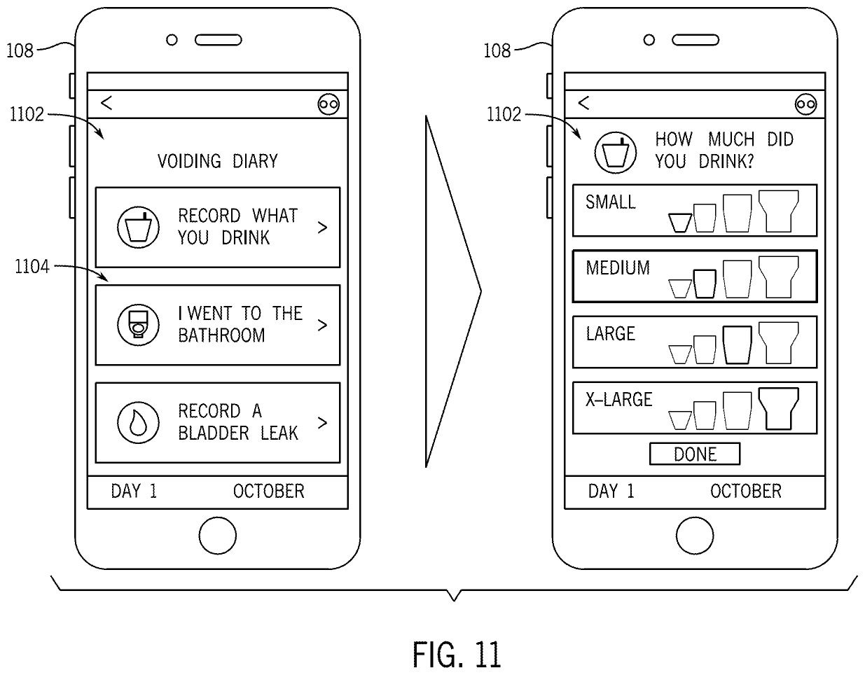

Examples of non-compliance issues 702 are shown. Some examples may include triggers 704 where the patient has not used the voiding device 200 in a certain amount of time (e.g., 24 or 48 hours), that the patient is using the voiding device 200 but not the voiding diary (discussed with respect to FIG. 11), or that the voiding device 200 has not been used within a given time of the user 110 waking up. See, e.g., triggers 704A, 704B, and 704C, prompting the user to use the device after waking up.

The triggers 704 may be used by the treatment management system 100 to determine when to take action to generate a message 706 to the user 110. For example, as discussed with respect to the operation 518, the treatment management system 100 may generate a message to the user 110 based on the message 706 corresponding to the particular non-compliance issue. The treatment management system 100 may also prompt the healthcare provider 106 to take a provider action 708 also associated with the particular non-compliance issue 702. For example, related non-compliance issues 702, triggers 704, messages 706, and provider actions 708 are shown in the rows of the table in FIG. 7. Any of the messages 706 may be customized for a particular user 110, study, intervention, and/or LUTS condition. The treatment management system 100 may generate one or more messages 706 to the user 110 based on the non-compliance issue 702 and/or the trigger 704. The system 100 may aggregate records of non-compliant users and present the records in a user interface, as seen for example in FIG. 16.

FIG. 8 is similar to FIG. 7 but shows a patient study schedule structure 800. Rather than being driven by patient non-compliance like the patient compliance response structure 700, the patient study schedule structure 800 is driven by the day of a void study that the user 110 has been prescribed by the healthcare provider 106. The patient study schedule structure 800 includes study days 802 and related triggers 804, messages 806, and provider actions 808. For example, related study days 802, triggers 704, messages 706, and provider actions 708 are shown in the rows of the table in FIG. 8. Any of the messages 706 may be customized for a particular user 110, study, intervention, and/or LUTS condition. For example, the messages 806 may include one or more placeholders 810 that the treatment management system 100 can fill in with relevant patient information. The treatment management system 100 may generate one or more messages 806 to the user 110 based on the study day 802 and/or the trigger 804.

A message may be associated with a study day, 802, a trigger 804, a message template 806 and a provider action 808. The study day 802 may be a day since the start of a study that the message template 806 is available to be sent to the user. The trigger 804 may be an action, or inaction, by the user 110 that causes the system 100 to generate the message. The message template may be default text, media, or information that may be sent to the user. The message template may be customizable with the user name, specific user instructions, or feedback tailored to the user. A provider action 808 may be associated with the message, directing the provider to take an action related to the user's use of the voiding device. For example the system 100 may generate a message 812 on day 1 of a voiding study. The message 812 may be triggered by a trigger 804 after the first void recorded on the first day of the trial. The template 806 may include sample introductory information that may be customized by placing information in a placeholder 810. The provider action 808 may indicate that the patient is instructed to use the voiding device and that the user data associated with the void is available to be reviewed by the provider 106.

FIG. 9 illustrates an example of a user study selection interface 900 suitable for use with the system of FIG. 1. In the example shown, the study selection interface 900 includes a study selection region 902 suitable to enable a healthcare provider 106 to select one or more studies such as the studies 904a-f. More or fewer studies may be available for selection. The study selection interface 900 may include a user metadata region 906 that displays metadata related to a particular user (e.g., patient record number or patient identifier, name, gender, age, and/or date of birth). The study selection interface 900 may include a date region 908 that displays the current date and/or time. The study selection interface 900 may include a help button 910 that opens a help screen for the healthcare provider 106. The study selection interface 900 may include a navigation button 912 that directs the healthcare provider 106 to another portion of a user interface of the treatment management system 100.

The one or more studies 904a-f may be selected based on baseline patient data, such as received in operation 502 of the method 500. A study may be customized or tailored to certain LUTS conditions and/or a user's particular medical history. User instructions may be tailored to a study such as described with respect to operations 504 and 504 described with respect to FIG. 5. The user instructions may be configured such that the user data collected via the study is configured to diagnose or treat a given condition. For example a study 904a may be configured to diagnose OAB and user instructions may be configured to gather user data related to OAB symptoms.

For example a study may collect patient data from the voiding device 200 suitable to enable the healthcare provider 106 to differentiate between OAB and BPH. These conditions may present similar symptoms and may be misdiagnosed as one another. Gathering accurate data via the voiding device 200 as described with respect to the methods 500 and/or 600 may enable the healthcare provider 106 to more accurately diagnose the appropriate condition and thus prescribe an appropriate treatment.

In another example, a study may be tailored to determine the efficacy of a treatment, such as a treatment prescribed in operation 406 of the method 400. For example, a study may be tailored to collect patient data with a voiding device 200 before treatment, during treatment, and/or post-treatment. The treatment management system 100 may compare pre-and post-treatment data and help a healthcare provider 106 determine the treatment efficacy. The healthcare provider 106 may, based on the comparison of pre-treatment data, data collected during treatment, and/or and post-treatment patient data, prescribe additional treatment, the cessation of treatment, and/or another treatment.

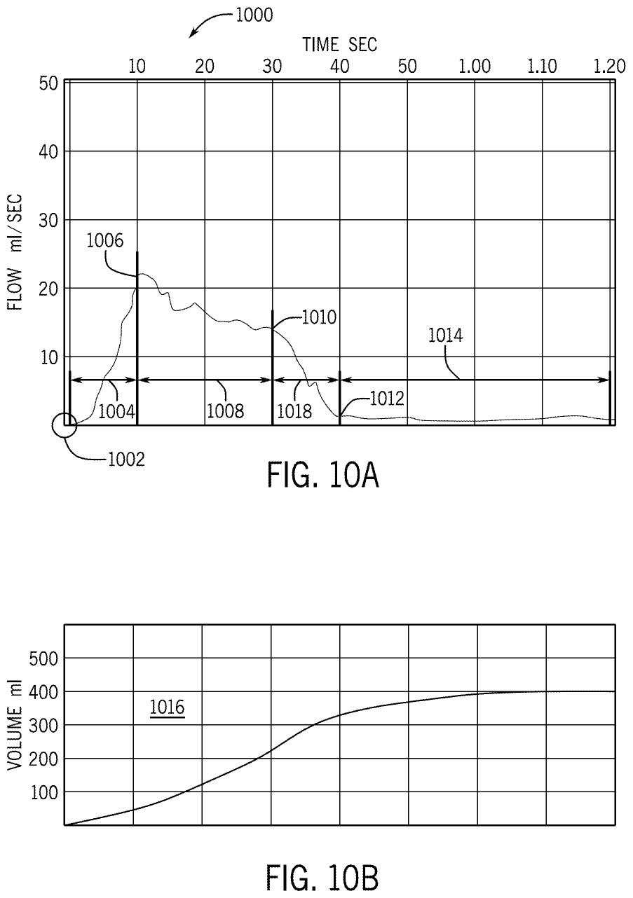

FIG. 10A and FIG. 10B illustrate an example of a void profile 1000. A void profile 1000 may be generated by the user 110 following the user instructions generated in the methods 500 and/or 600. For example when the system 100 generates user instructions in operation 506, the user 110 may use the voiding device and generate user data in operation 508 and/or 608. The user data may, in many embodiments, include the voiding profile 1000. The user data may include other data such as date, time, location, user identifier, a study identifier, or the like. A processing element of the user device 108, the healthcare provider device 104, the voiding device 200, and/or the server 102 may use the inlet flow of urine over time, and total urine volume accumulated over time, to develop a void profile. An example of a void profile may be seen in FIG. 10A and FIG. 10B. FIG. 10A illustrates an example of urine flow over time. FIG. 10B illustrates an example of accumulated urine flow over time. As illustrated in FIG. 10A, the void profile 600 may have points and regions that may be detected by the respective processing element of the user device 108, the healthcare provider device 104, and or the server 102. For example, a point may be a single point in time. A region may span between two or more points. The void profile is generated by the respective processing element by plotting the determined flow rate over the flow event time frame. Additionally, determined volume vs. time relationships may be plotted.

Generally, the void profile 1000 may further have a region of increasing urine flow. For example, the void profile 1000 may have a time to maximum flow 1004 where urine flow rate increases from the point of onset of urination 1002 to a maximum value at maximum point 1006. At maximum point 1006, urine flow may decrease, but is variable depending on the particular patient.

The void profile 1000 may further have a region of slowly decreasing urine flow. For example, void profile 1000 may have a region 1008 where urine flow rate decreases slightly over time from the maximum point 1006 or where urine flow rate remains substantially uniform over time. Urine flow rate may continue in region 1008 to point 1010, the beginning of the terminal region of urination. At point 1010, the time rate of change of the flow rate of urine may begin to decrease in region 1018, relative to region 1008. In region 1018, the flow rate of urine may decrease to a point 1012 where urine flow rate substantially ceases. Following the point of cessation of urination, e.g., at point 1012, the void profile 1000 may include an additional region 1014. Region 1014 may represent a delay while the voiding device 200 waits to determine if urination may begin again. Some urinary health problems involve halting urination, where urine flow starts and stops multiple times with in a single void event. Region 1014 in void profile 1000 will help the voiding device 200 capture urination data consistent with such problems. As described with respect to operation 412 of method 600, the server processing element 152 or the device processing element 252 may numerically integrate the flow rate of urine over time to develop an accumulated urine profile, for example profile 1016.

FIG. 11 illustrates an example of an electronic voiding diary 1102 suitable for use with the system of FIG. 1. The voiding diary 1102 may be executed as an application on the user device 108. The voiding diary 1102 may include one or more user interface screens that prompt a user 110 to input information related to the user's voiding, fluid intake, and the like. The voiding diary 1102 may include a qualitative data input region 1104 adapted to receive qualitative data related to the voiding behavior of the user 110. For example, the qualitative data input region 1104 may be adapted to collect data related to the user's fluid intake, bathroom usage, and/or bladder leakage. The voiding diary 1102 may be adapted to collect information related to the amount of a user's fluid intake. The qualitative user data may be related to one or more of total volume of urine output, fluid intake, bladder leaks, bedtime, or awake time.

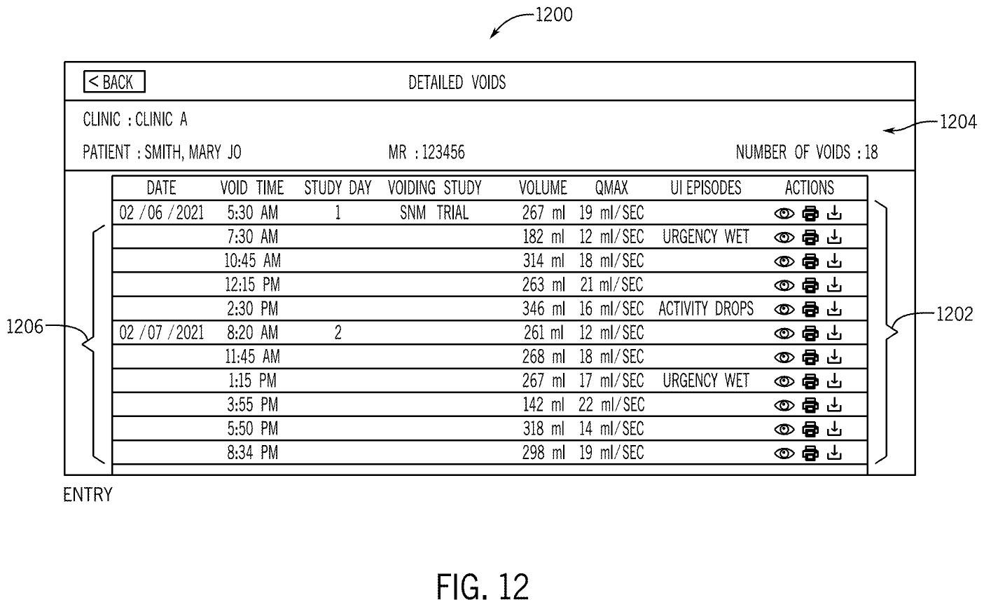

FIG. 12 illustrates a voiding record 1200 that summarizes void data from a user 110 such as may be collected by a voiding device 200 or voiding device 300. The voiding record may be an aggregation of one or more voiding profiles 1000 or other user data captured in operations 508 and/or 608 of the methods 500 and/or 600. The voiding record 1200 may provide an aggregated view of the user's voids to facilitate diagnosis of the user's LUTS condition, the efficacy of a treatment, or the like. The voiding record 1200 may include one or more entries 1206 with detailed void data from the user 110. The entries 1206 may include a date and time of the void, a study day (such as a study day 802 of the patient study schedule structure 800), a voiding study type (such as a study 904a-f), a void volume (e.g., 267 mL), maximum flowrate, and data related to urinary incontinence (e.g., urine leakage events such as may be recorded by the voiding diary 1102). One or more provider actions 1202 may be provided for an entry 1206. For example, the healthcare provider 106 may be able to see more detail, print the entry 1206, and/or download the data in the entry 1206. The voiding record 1200 may include a user metadata region 1204 similar to the user metadata region 906 of the study selection interface 900.

With reference to FIG. 13, the treatment management system 100 may generate one or more voiding studies 1300. A voiding study 1300 may be tailored to a particular intervention, user 110, or LUTS condition. The voiding study 1300 may have a region that displays baseline data 1302, such as for example baseline patient data collected by a voiding device 200 in the operation 402 and/or the operation 502. The voiding study 1300 may include a post-intervention data 1304 and/or a post-intervention data 1306 collected by a voiding device 200 in an operation 408. The voiding study 1300 may include one or more sets of user data including one or more void profiles 1000 captured during treatment. The baseline data 1302, post-intervention data 1304, and/or post-intervention data 1306, and or data captured during treatment may be used by a healthcare provider 106 to determine a diagnosis of the LUTS condition of the user 110 and/or determine the efficacy of an intervention. The voiding study 1300 may include a user metadata region 1308 similar to the user metadata region 906 of the study selection interface 900. The voiding study 1300 may include a patient data history region 1310 that shows patient data related to voiding at one or more time periods. For example, the patient data history region 1310 may display a voided volume, voiding time, flow time, time to maximum flow (see, e.g., the time to maximum flow 1002 in FIG. 10A). The voiding study 1300 may include a patient data summary region 1312 that summarizes patient data. The voiding study 1300 may include a patient data change region 1314 that shows changes in patient data over time. The patient data change region 1314 may be beneficial for a healthcare provider 106 to determine whether an intervention is helping to improve the user's LUTS condition.

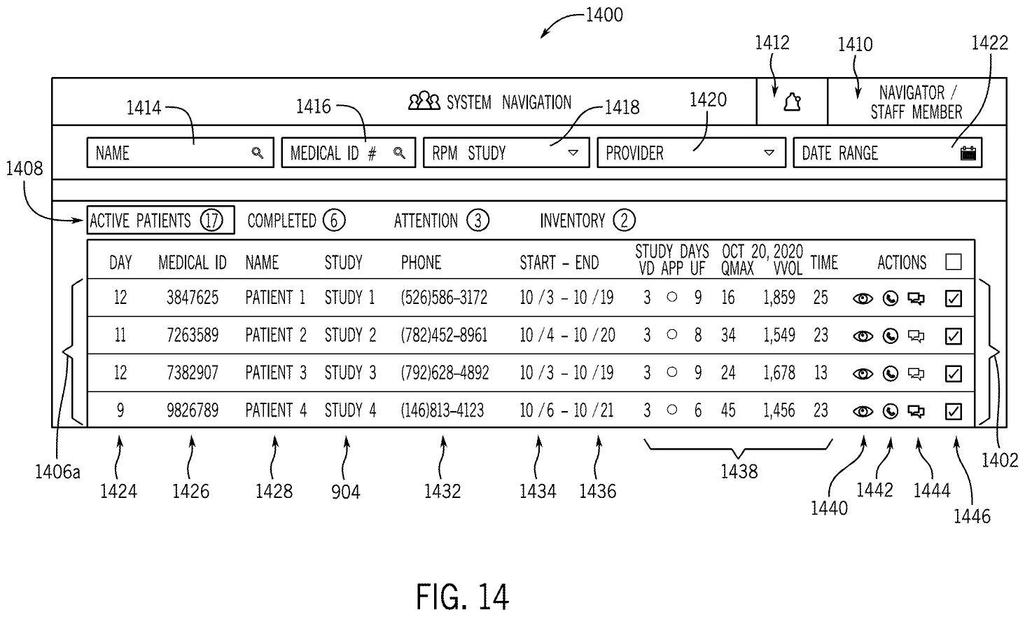

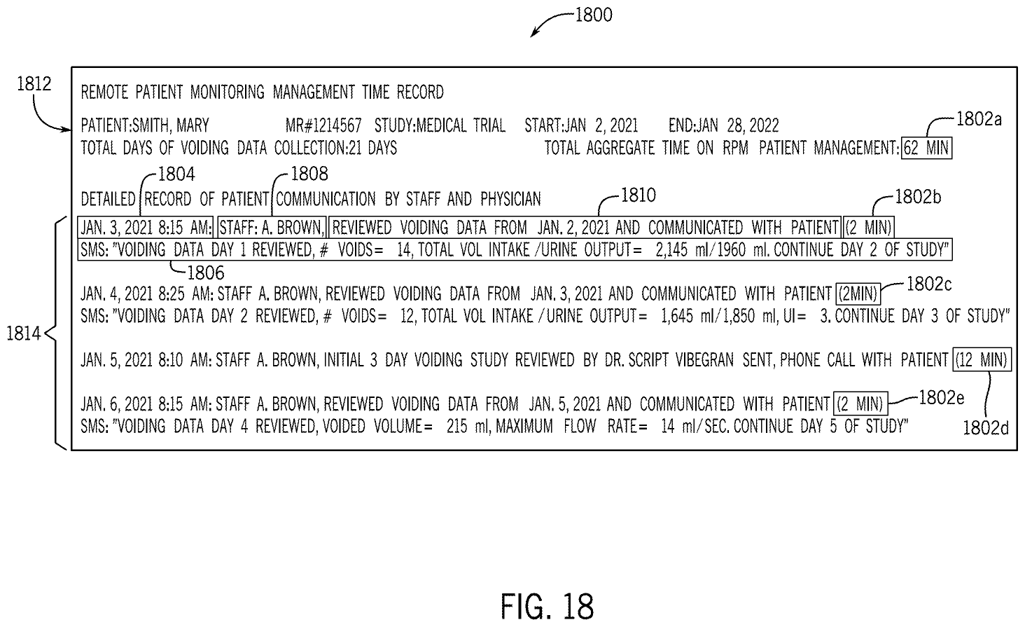

With reference to FIG. 14-FIG. 17, a navigation interface of the treatment management system 100 is shown. The navigation interface may provide for the retrieval, review, manipulation, reporting, summarization, and/or storage of user data for one or more patients. The navigation interface may be expandable to show all the data collected for a user, and associated actions taken by the user and/or provider, as well as the user's course of treatment, if applicable. The navigation interface may display a collective assembly of the same data for many patients, resulting in the efficient management of all the patients and their respective courses of treatment and/or status. The navigation interface 1400 may include a user metadata region 1404 similar to the user metadata region 906 of the study selection interface 900. The navigation interface 1400 may include a report selection region 1408 that enables a healthcare provider 106 to select different entries for display. For example, the report selection region 1408 may enable the healthcare provider 106 to select entries related to active users, users who have completed use of a voiding device, users flagged by the treatment management system 100 for attention from the healthcare provider 106, and/or an inventory of the voiding devices. The navigation interface 1400 may display, as shown in FIG. 14, one or more entries 1406a when active patients are selected. Active patients may be a user or users 110 who are actively participating in a voiding study 1300 or otherwise engaged with the treatment management system 100. The navigation interface 1400 may also display a user or users 110 that once were active patients (past active patients) but that have completed a course of treatment and/or voiding study, which information may be displayed together with other currently active patients or separate from currently active patients. The navigation interface 1400 may also display one or more entries 1406b corresponding to a patient or patients who have completed a voiding study or other interaction with the system 100, entries 1406c corresponding to a user or users who are non-compliant with user instructions, such as users for whom a communication may be generated in operation 518 and/or 610 of methods 500 or 600, respectively, and/or entries 1406d corresponding to an inventory of voiding device and/or related supplies, described further herein. The one or more entries 1406a may include patient data related to user 110 voiding. For example, an entry 1406a may include a study day 1424; user 110 patient identifier 1426; user 110 name 1428; a study 904 assigned to the user 110; the user 110 contact information 1432 such as phone number, email address, physical address or the like; study start date 1434 and/or end date 1436; study data 1438 such as use of the voiding diary 1102 and/or voiding device 200; maximum urine flow; urine volume; remote patient monitoring (“RPM”) time (discussed in more detail with respect to FIG. 18); and/or provider actions 1402.

The user metadata region 1404 may additionally enable a healthcare provider 106 to filter, sort, or otherwise select one or more entries 1406a. For example, the user metadata region 1404 may include a user 110 name selection field 1414 that enables a healthcare provider 106 to select one or more entries 1406a based on a user 110 name. The user metadata region 1404 may include a medical identification selection field 1416 that enables a healthcare provider 106 to select one or more entries 1406a based on a user 110 patient identifier. The user metadata region 1404 may include a remote patient monitoring study selection field 1418 that enables a healthcare provider 106 to select one or more entries 1406a based on a user 110 RPM study (discussed in more detail with respect to FIG. 18). The user metadata region 1404 may include a provider selection field 1420 that enables a healthcare provider 106 to select one or more entries 1406a based on a healthcare provider 106. The user metadata region 1404 may include a date selection field 1422 that enables a healthcare provider 106 to select one or more entries 1406a based on a date range of patient data. The navigation interface 1400 may include an alert indicator 1412 configured to notify a healthcare provider 106 of issues that may benefit from the attention of the healthcare provider 106. The navigation interface 1400 may include a healthcare provider 106 designation region 1410 configured to display the name or other information associated with a healthcare provider 106 using the navigation interface 1400. One or more of the name selection field 1414, medical identification selection field 1416, remote patient monitoring study selection field 1418, provider selection field 1420, date selection field 1422 may enable a provider to filter user records stored in the system 100 to easily navigate to desired individual users, groups of users, providers, or the like. The data in the field may be linked to one another such that a selection in one field automatically updates the available data in the other fields. For example, if Provider A is selected in the field 1420, the user field 1414 may be updated to only show the names of Provider A's patients. Similarly, the entries 1406a may be filtered or sorted base on the selections in the fields 1414, 1416, 1418, 1420, and/or 1422.

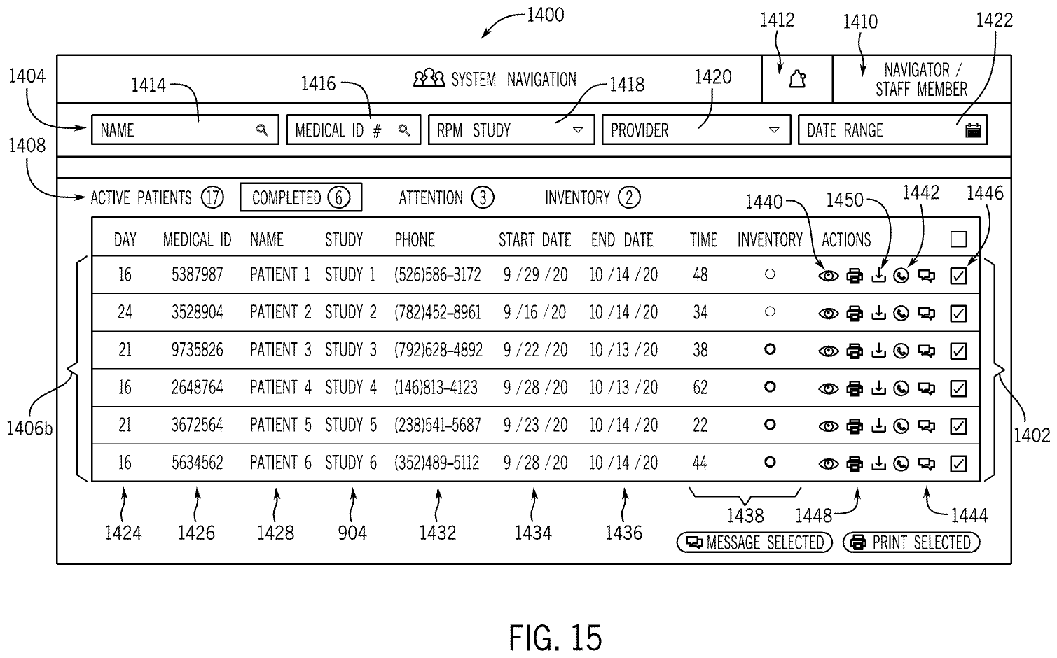

The navigation interface 1400 may include or associate one or more provider actions 1402 with the one or more entries 1406a, entries 1406b, entries 1406c, and/or entries 1406d. For example, provider actions 1402 may be accessed via one or more user interface elements to enable a healthcare provider 106 to see more detail on an entry 1406a (e.g., via user interface element 1440), call a user 110 (e.g., via user interface element 1442), send a message (such as a message 706 and/or message 806 via a user interface element 1444) to a user 110, and/or select a record (e.g., via a user interface element 1446). Respective user interface elements 1448 and 1450 may be provided to enable the provider to print and/or download data associated with an entry 1406a-d (see, e.g., FIGS. 15-17). The same user interface elements 1440, 1442, 1444, 1446, 1448, and/or 1450 may be displayed on each of the configurations of the navigation interface 1400. A message may include a text message, email, pre-recorded voice message, video and/or audio content. The treatment management system 100 may send a message to the user device 108 from the navigation interface 1400, such as in response to the user's noncompliance with user instructions discussed with respect to the methods 500 and/or 600. In some implementations, the treatment management system 100 may send a message and/or direct a telephone call directly from the navigation interface 1400 to the user device 108. Provider actions 1402 may be provided to print, download, or select one or more entries 1406a, entries 1406b, entries 1406c, and/or entries 1406d.

FIG. 15 illustrates the navigation interface 1400 configured to display one or more entries 1406b related to users 110 who have completed a voiding study 1300 with the treatment management system 100. The entries 1406b include many of the same fields of information as the entries 1406a.

FIG. 16 illustrates the navigation interface 1400 configured to display one or more entries 1406c related to users 110 whom may benefit from attention from a healthcare provider 106. For example, a user 110 whom the treatment management system 100 has determined is not following the provider instructions for a voiding study 1300, such as may be determined in the method 500, may be listed in the one or more entries 1406c. The entries 1406c include many of the same fields of information providing the same or similar functions as the entries 1406a and/or entries 1406b. For example, the entries 1406b and/or 1406c may include one or more of the following: a study day 1424; user 110 patient identifier 1426; user 110 name 1428; a study 904 assigned to the user 110; the user 110 contact information 1432 such as phone number, email address, physical address or the like; study start date 1434 and/or end date 1436; study data 1438 such as use of the voiding diary 1102 and/or voiding device 200; maximum urine flow; urine volume; remote patient monitoring (“RPM”) time; and/or provider actions 1402. The entries 1406c may include comment data from the healthcare provider 106.

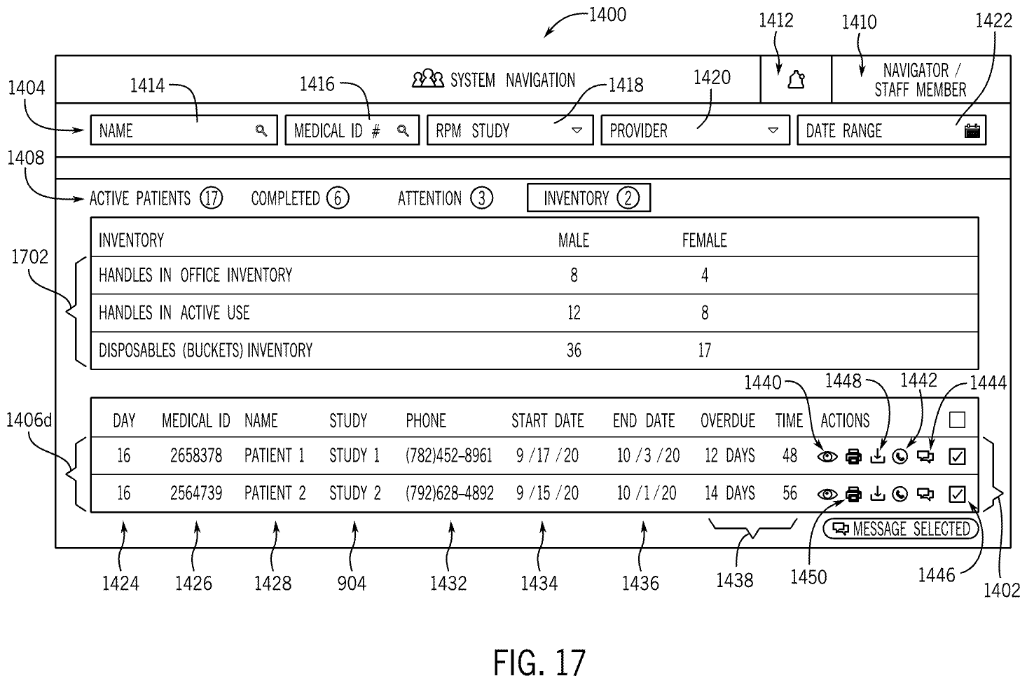

FIG. 17 illustrates the navigation interface 1400 configured to display an inventory region 1702 including data of voiding devices and associated components. For example, the inventory region 1702 may display handles (e.g., handles 202, 302) in use by users 110, handles in inventory, and/or flow chambers or buckets 204, 304. The inventory region 1702 may include different information than displayed in the entries 1406a-d shown in FIGS. 14-17. Such control and display of inventory of voiding devices may enable the treatment management system 100 to assure that an adequate supply of handles and/or voiding chambers are available for a current or prospective user 110 demand. Management of inventory may be facilitated by the RFID component 360 in the voiding device. For example, a handle may be checked-out to a user 110 by scanning the RFID component 360 with a device in communication with the treatment management system 100. Similarly, the handle may be returned to the provider, dis-associated with a user, sanitized, recharged, and/or placed back in service to be available for a new user. A benefit of the inventory region 1702 may be that the treatment management system 100 may be able to automatically anticipate and/or fulfill handle and/or bucket orders from healthcare providers 106. The provider may be able to see in one screen the devices in use and in inventory and thus manage the inventory by ordering more devices as needed. For example, the treatment management system 100 may-based on past usage of the voiding devices, inventory level, and/or patient numbers-predict when a healthcare provider 106 should be sent additional handles, buckets, or other related equipment.

FIG. 18 illustrates an example of an RPM record 1800. The RPM record 1800 includes a user metadata region 1812 similar to the user metadata region 906 and others disclosed herein. The RPM record 1800 includes one or more entries 1814 that include information about interactions between the user 110 and the healthcare provider 106. For example, an entry 1814 may include date/time data 1804 indicative of the date and/or time of the interaction (e.g., “Jan. 3, 2021 8:15 AM”). The entry 1814 may include provider data 1808 indicative of the healthcare provider 106 who interacted with the user 110 (e.g., “A. Brown”). The entry 1814 may include one or more provider comments 1810 that may include notes or observations of the healthcare provider 106 with the user 110 (e.g., “reviewed voiding data” or the like). The entry 1814 may include message data 1806 including messages sent to the user 110, such as to the user device 108 by the treatment management system 100. The message data 1806 may include patient data acquired by a voiding device or another sensor device (e.g., “Voids=14, total fluid intake/urine output =1,645 mL/1850 mL” or the like). The message data 1806 may include one or more next actions recommended by the healthcare provider 106 for the user (e.g., “continue day #2 of the study”).

The collection of the content of the RPM record as created by the system 100 based on the activities may provide the basis for recording time spent on activities to manage workloads, work flow, resource allocation, and provide an auditable report of entries 1406a-d associated with a user voiding study, void profile, and/or other interactions between the user, the provider, and or the system 100. The amount of time the healthcare provider 106 spends interacting with the user 110 may be recorded in one or more RPM time entries 1802, such as RPM time entries 1802a-e. An RPM time entry, and the RPM record 1800 may be used advantageously by the healthcare provider 106 for billing purposes. With prior art treatment management systems, time spent remotely monitoring and managing patient health may often go un-accounted for and is thus not often able to be claimed for reimbursement from a payer, such as an insurance company, the user 110, a third party, Medicare, and/or Medicaid. The treatment management system 100 solves this problem by documenting how the healthcare provider 106 has interacted with the user 110 and how much time that interaction took. The payer may be billed according to the RPM time entries 1802. The treatment management system 100 may automatically generate a bill payable by the payer. One or more RPM time entries may include an aggregate or sum of time entries for individual interactions with the user 110. For example, the RPM time entry 1802a may indicate a total amount of RPM time spent with a user, such as a total of the 1802b-e and/or other RPM time entries. The RPM record 1800 may also be advantageous for reporting, auditing, government compliance, and other purposes.

FIG. 19 is an example of a navigation interface 1900 of the system of FIG. 1. The navigation interface 1900 may include one or more fields suitable to manage one or more patients through their treatment journey similar to other navigation interfaces disclosed herein. The navigation interface 1900 may be a more efficient and intuitive interface for displaying and manipulating patient information. The navigation interface 1900 visually shows the status of one or more patients and key actions that may be required for treatment. The navigation interface 1900 may include a menu 1934 configured to allow a user to select different roles within the treatment management system 100. For example, the menu 1934 may be disposed as a sidebar on the left portion of the navigation interface 1900. The menu 1934 may be configured differently for specific user roles. The menu 1934 may allow the user to dig deeper into a study status, inventory management, reporting, access to patient records and related details, and access to customer-related data.

The navigation interface 1900 may include a health system designation field 1902 suitable to select a health system whose patients are to be managed. The navigation interface 1900 may include a clinic designation field 1904 suitable to select a clinic within the health system selected via the health system designation field 1902. The navigation interface 1900 may include one or more patient entries 1906. The one or more patient entries 1906 may include one or more fields of information related to a patient treatment. For example, an entry 1906 may include a patient identifier 1908 that uniquely identifies a patient. An entry 1906 may include a patient name indicator 1912 that indicates a patient name. An entry 1906 may include a study type indicator 1914 that indicates a study type. An entry 1906 may include a study progress indicator 1918 that indicates a patient's progress on a study journey. An entry 1906 may include a remote patient monitoring indicator 1920 that indicates time accumulated by a provider remotely monitoring a patient. An entry 1906 may include an appointment date indicator 1922 that indicates a date of a next patient appointment with a provider. An entry 1906 may include a wakeup time indicator 1924 that indicates a time for a wakeup alarm for the patient. The navigation interface 1900 may include a text message indicator 1926 that indicates a number of messages sent to a patient. The navigation interface 1900 may include a call indicator 1928 that indicates a number of calls to a patient. The navigation interface 1900 may include an assessment indicator 1930 that indicates a progress of a patient assessment. The navigation interface 1900 may include an active study indicator 1932 that indicates a number of active patient studies in progress.

The navigation interface 1900 may beneficially present easily-understood icons suitable to facilitate common understanding across navigators. The navigation interface 1900 may be useable without a significant amount of user education prior to using the navigation interface 1900.

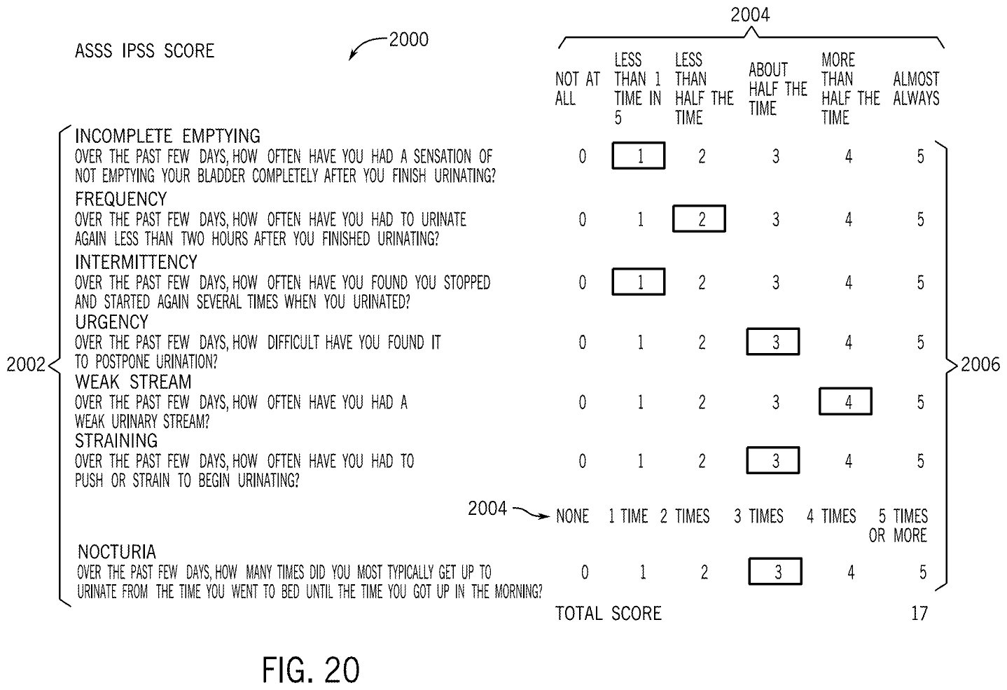

FIG. 20 shows an example of a voiding record 2000. The voiding record 2000 may include one or more assessment regions 2002. The voiding record 2000 may include one or more fields that enable a patient to indicate the occurrence of urinary incontinence issues. For example, the assessment region 2002 enable a patient to indicate the frequency of incomplete bladder emptying, frequency of urination, intermittency of urination, urgency of urination, weakness of urine stream, straining to begin urination, and/or urination that interrupts sleep (e.g., nocturia).

The voiding record 2000 may include one or more frequency selectors 2004 that enable a patient to indicate a frequency of the incontinence issues indicated in the assessment region 2002. The voiding record 2000 may include one or more scoring regions 2006 that enable the patient to score the frequency or occurrence of incontinence issues. For example, the scoring region 2006 may indicate a numerical score (e.g., 0-5) that indicates the frequency or occurrence of incontinence issues such as indicated in the assessment region 2002.

The voiding record 2000 may be utilized when a patient is initiated by the provider in a patient journey. The voiding record 2000 may digitize the capture of urinary incontinence issues and seamlessly integrate the related data into the patient disease state management journey. The voiding record 2000 may provide the benefit of easily or more accurately capturing incontinence symptom data.

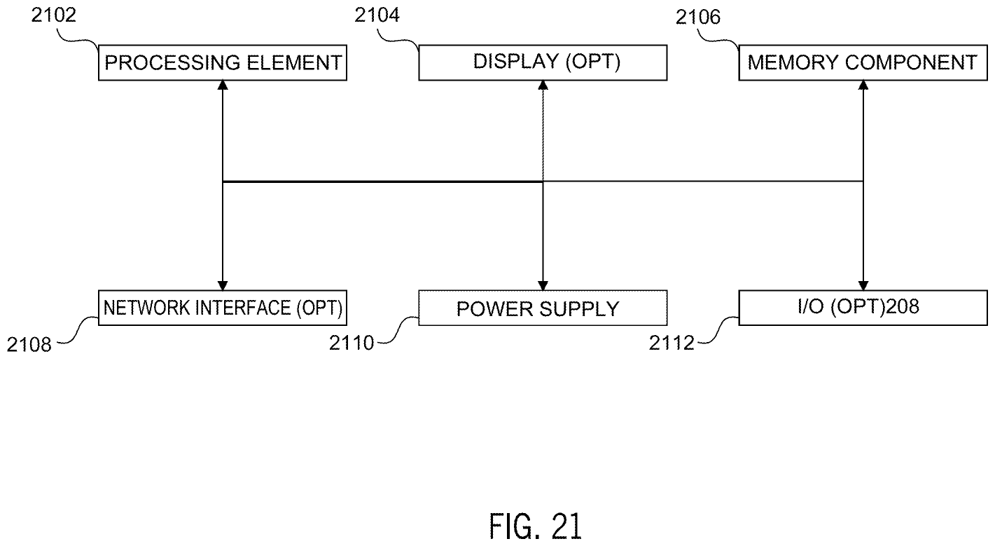

FIG. 21 illustrates a simplified block diagram for the various devices of the system 100 including the server 102, the user device 108, the healthcare provider device 104, the voiding device 200, and/or the voiding device 300. As shown, the various devices may include one or more processing elements 2102, an optional display 2104, one or more memory components 2106, a network interface 2108, optional power supply 2110, and an optional input/output I/O interface 2112, where the various components may be in direct or indirect communication with one another, such as via one or more system buses, contract traces, wiring, or via wireless mechanisms.

The one or more processing elements 2102 may be substantially any electronic device capable of processing, receiving, and/or transmitting instructions. For example, the processing elements 2102 may be a microprocessor, microcomputer, graphics processing unit, or the like. It also should be noted that the processing elements 2102 may include one or more processing elements or modules that may or may not be in communication with one another. For example, a first processing element may control a first set of components of the computing device and a second processing element may control a second set of components of the computing device where the first and second processing elements may or may not be in communication with each other. Relatedly, the processing elements may be configured to execute one or more instructions in parallel locally, and/or across the network, such as through cloud computing resources.

The display 2104 is optional and provides an input/output mechanism for devices of the system 100, such as to display visual information (e.g., images, graphical user interfaces, videos, notifications, and the like) to a user, and in certain instances may also act to receive user input (e.g., via a touch screen or the like). The display may be an LCD screen, plasma screen, LED screen, an organic LED screen, or the like. The type and number of displays may vary with the type of devices (e.g., smartphone versus a desktop computer).

The memory components 2106 store electronic data that may be utilized by the computing devices, such as audio files, video files, document files, programming instructions, and the like. The memory components 2106 may be, for example, non-volatile storage, a magnetic storage medium, optical storage medium, magneto-optical storage medium, read only memory, random access memory, erasable programmable memory, flash memory, or a combination of one or more types of memory components. In many embodiments, the server 102 may have a larger memory capacity than the user device 108, with the memory components optionally linked via the network 112 or the like.

The network interface 2108 receives and transmits data to and from the network 116 to the various devices of the system 100. The network interface 2108 may transmit and send data to the network directly or indirectly. For example, the networking/communication interface may transmit data to and from other computing devices through the network 116. In some embodiments, the network interface may also include various modules, such as an application program interface (API) that interfaces and translates requests across the network 112 to the specific server 102, voiding device 200, 300, user device 108, or healthcare provider device 104. The network interface 2108 may be any suitable wired or wireless interface. For example, the network may be an Ethernet network, Wi-Fi, Bluetooth, WI-Max, Zigbee network, the internet, microwave link, or the like.

The various devices of the system may also include a power supply 2110. The power supply 2110 provides power to various components of the server 102, user device 108, voiding devices 200, 300, or healthcare provider device 104. The power supply 2110 may include one or more rechargeable, disposable, or hardwire sources, e.g., batteries, power cord, AC/DC inverter, DC/DC converter, or the like. Additionally, the power supply 2110 may include one or more types of connectors or components that provide different types of power to the user device 108, healthcare provider device 104, voiding devices 200, 300, and/or server 102. In some embodiments, the power supply 2110 may include a connector (such as a universal serial bus) that provides power to the computer or batteries within the computer and also transmits data to and from the device to other devices.

The I/O interface 2112 allows the system devices to receive input from a user and provide output to a user. In some devices, for instance the remote computing device 108, the I/O interface 2112 may be optional. For example, the I/O interface 2112 may include a capacitive touch screen, keyboard, mouse, stylus, or the like. The type of devices that interact via the input/output interface 140 may be varied as desired.

The description of certain embodiments included herein is merely exemplary in nature and is in no way intended to limit the scope of the disclosure or its applications or uses. In the included detailed description of embodiments of the present systems and methods, reference is made to the accompanying drawings which form a part hereof, and which are shown by way of illustration specific to embodiments in which the described systems and methods may be practiced. These embodiments are described in sufficient detail to enable those skilled in the art to practice presently disclosed systems and methods, and it is to be understood that other embodiments may be utilized, and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The included detailed description is therefore not to be taken in a limiting sense, and the scope of the disclosure is defined only by the appended claims.

From the foregoing it will be appreciated that, although specific embodiments of the invention have been described herein for purposes of illustration, various modifications may be made without deviating from the spirit and scope of the invention.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Of course, it is to be appreciated that any one of the examples, embodiments or processes described herein may be combined with one or more other examples, embodiments and/or processes or be separated and/or performed amongst separate devices or device portions in accordance with the present systems, devices and methods.

Finally, the above discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

1-8. (canceled)

9. A method for treating lower urinary tract symptoms comprising:
receiving, by a uroflowmeter, first user data related to a urinary characteristic of a user, wherein the first user data is collected prior to the user receiving an intervention for the lower urinary tract symptoms;
determining, by a processing element, a user study including a monitoring protocol, based at least in part on the first user data;
receiving, from the uroflowmeter, second user data related to the urinary characteristic of the user collected in response to the monitoring protocol;
comparing, by the processing element, the second user data relative to the monitoring protocol;
determining, by the processing element, a difference based on the comparison;
generating, by the processing element, a communication related to the difference; and
transmitting, by the processing element, the communication to a user device associated with the user.

10. The method of claim 9, wherein the communication is configured to prompt the user to follow the monitoring protocol.

11. The method of claim 9, further comprising:
adjusting the monitoring protocol based on the difference; and/or
determining the monitoring protocol based on the lower urinary tract symptoms.

12. (canceled)

13. The method of claim 9, further comprising:
determining, by the user device, qualitative user data associated with the lower urinary tract symptoms; and
receiving, by the processing element, the qualitative user data.

14. The method of claim 13, wherein the qualitative user data is related to one or more of total volume of urine output, fluid intake, bladder leaks, bedtime, or awake time.

15. The method of claim 13, further comprising generating a voiding study including the qualitative user data, the first user data, and the second user data.

16. The method of claim 15, further comprising determining the intervention configured to treat the lower urinary tract symptoms based on the voiding study.

17. The method of claim 16, wherein:
the intervention is one of a first level intervention, a second level intervention, or a third level intervention;
the second level intervention is more invasive to a body of the user than the first level intervention; and
the third level intervention is more invasive to the body of the user than the second level intervention.

18. The method of claim 9, wherein the urinary characteristic is one or more of a peak urine flow, a time to the peak urine flow from an onset of a urine flow, a urine volume, or a urination time.

19. The method of claim 9, further comprising generating a remote patient monitoring (“RPM”) record including an entry related to an interaction between the user and a medical provider.

20. The method of claim 19, wherein the entry comprises an RPM time entry configured to track a time of the interaction and generating a bill payable by a payer based on the RPM time entry.

21. (canceled)

22. The method of claim 9, wherein the uroflowmeter further comprises:
a handle portion adapted to be gripped by the user to position the uroflowmeter for the collection of urine from the user; and/or
electronics that determine a fill volume of the flow chamber using a movement of a magnet; and/or
a flow chamber that defines an inlet that receives a flow of urine and an outlet that evacuates urine from the flow chamber at a predetermined rate.

23. The method of claim 22, wherein the uroflowmeter further comprises an arm, a magnet, a sensor and a float, wherein:
the arm connects the float and the magnet;
the arm and the magnet are connected to one another about a pivot axis;
the magnet rotates about the pivot axis in response to movement of the float; and
the sensor further detects a change in an angular position of the magnet, wherein movement of the magnet is correlated to the first user data.

24-30. (canceled)

31. A method for treating lower urinary tract symptoms comprising:
receiving, by a uroflowmeter, first user data related to a urinary characteristic of the user, wherein the first user data is collected prior to the user receiving an intervention for the lower urinary tract symptoms;
determining a cause of the lower urinary tract symptoms;
determining, based on the cause, the intervention for the lower urinary tract symptoms; and
receiving, from the uroflowmeter, second user data related to the urinary characteristic of the user collected in response to a monitoring protocol, wherein the second user data is collected after the user begins receiving the intervention.

32. The method of claim 31, wherein:
the intervention is one of a first level intervention, a second level intervention, or a third level intervention;
the second level intervention is more invasive to a body of the user than the first level intervention; and
the third level intervention is more invasive to the body of the user than the second level intervention.

33. The method of claim 31, further comprising:
receiving, from the uroflowmeter, third user data related to the urinary characteristic of the user; and
determining an efficacy of the intervention.

34-35. (canceled)

36. A system for treating lower urinary tract symptoms comprising:
a uroflowmeter configured to collect first user data related to a urinary characteristic of a user, wherein the first user data is collected prior to the user receiving an intervention for the lower urinary tract symptoms;
a processing element associated with a medical provider device, wherein the processing element is configured to:
receive, from the uroflowmeter, second user data related to the urinary characteristic of the user collected in response to a monitoring protocol;
compare, by the processing element, the second user data relative to the monitoring protocol;
determine, by the processing element, a difference based on the comparison;
generate, by the processing element, a communication related to the difference; and
transmit, by the processing element, the communication to a user device associated with the user.

37. (canceled)

38. The system of claim 36, wherein the uroflowmeter comprises:
a handle portion adapted to be gripped by the user to position the uroflowmeter for a collection of urine from the user; and/or
a flow chamber configured to receive a flow of urine;
a buoyant float positioned within the flow chamber and positionable according to a urine level in the flow chamber,
a magnet associated with the buoyant float and positionable according to a position of the buoyant float, and
a sensor adjacent to the magnet and configured to detect a movement of the magnet, wherein the movement of the magnet is correlated to the first user data.

39. The system of claim 36, wherein;
the processing element is configured to generate a remote patient monitoring (“RPM”) record including an entry related to an interaction between the user and a medical provider and the entry comprises an RPM time entry configured to track a time of the interaction; and/or
the processing element is configured to generate a bill payable by a payer based on the RPM time entry.

40-42. (canceled)

43. The system of claim 36, wherein the processing element is further configured to:
receive the first user data; and
generate a user study, including the monitoring protocol, based at least in part on the first user data.
